(19) United States
(12) Patent Application Publication
(10) Pub. No.: US 2024/0140992 A1
(43) Pub. Date: May 2, 2024

(54) HEXADEPSIPEPTIDE COMPOUNDS AND METHODS OF USING THE SAME

(71) Applicants: Lodo Therapeutics Corporation, New York, NY (US); Zymergen Inc., Emeryville, CA (US)

(72) Inventors: Maria Diarey TIANERO-MCINTOSH, New York, NY (US); Arlene Atup SY-CORDERO, New York, NY (US); Bradley Morgan HOVER, New York, NY (US); Steven L. COLLETTI, New York, NY (US); Gayatri GOLLAPUDI, New York, NY (US)

(21) Appl. No.: 18/547,321

(22) PCT Filed: Feb. 22, 2022
(86) PCT No.: PCT/US2022/017263

§ 371 Date: Aug. 21, 2023

Related U.S. Application Data
(60) Provisional application No. 63/152,006, filed on Feb. 22, 2021.

(51) Int. Cl. C07K 11/02; C07K 14/36; C12N 9/00; C12N 9/10; C12N 15/52; C12N 15/76; C12P 21/02

(52) U.S. Cl. CPC C07K 11/02; C07K 14/36; C12N 9/1029; C12N 9/93; C12N 15/52; C12N 15/76; C12P 21/02; A61K 38/00

(57) ABSTRACT

The present disclosure provides compounds of Formula (I):

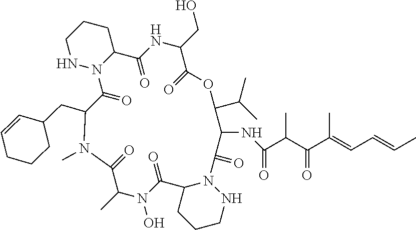 [SMILES: C/C=C/C=C(\C)C(=O)C(C)C(=O)NC1C(=O)N2NCCCC2C(=O)N(O)C(C)C(=O)N(C)C(CC2C=CCCC2)C(=O)N2NCCCC2C(=O)NC(CO)C(=O)OC1C(C)C]

or a pharmaceutically acceptable salt, solvate, or tautomer thereof, and uses of the same in treating a disease or disorder (e.g., cancer or fibrosis).

### RELATED APPLICATIONS

This application claims priority to U.S. Provisional Application No. 63/152,006, filed Feb. 22, 2021, the entire contents of which is incorporated herein by reference.

### INCORPORATION BY REFERENCE OF SEQUENCE LISTING

The present application is being filed with a Sequence Listing in electronic format. The Sequence Listing is provided as a file entitled ZYMR-50901WO_SeqList.txt, created on Feb. 22, 2022, and is 778 kilobytes in size. The information in electronic format of the Sequence Listing is incorporated by reference in its entirety.

### BACKGROUND

Many examples of semisynthetic derivatives of natural product therapeutics are derived and inspired from cyclic peptides, such as cyclosporine, daptomycin, romidepsin, aplidine, atosiban, caspofungin, telavancin, linaclotide, and pasireotide. These structurally diverse molecules are the active pharmaceutical ingredients of prescribed drugs to treat a range of indications across many therapeutic applications such as immunosuppressants, antibacterials, antifungals, oncology, premature birth, IBS, and Cushings syndrome. One minimally studied natural product cyclic peptide is verucopeptin, a cyclic hexadepsipeptide which has demonstrated antiproliferative activity with potential applications to oncology. Over the last 10-20 years, the search for new biologically-active bacterially-produced natural products using the classical culturing approach has frequently led to the re-discovery of the same metabolites. Recently, methodology has been revolutionized to develop biologically active congeners that arise from evolutionarily related biosynthetic gene clusters. These congeners are structurally related to natural products. These close analogs typically engage the same therapeutic target, yet they often exhibit differences in biological activity, including differences in potency and therapeutic index to general cytotoxicity.

There is unmet medical need for the discovery of new agents to treat various conditions, including cancer and fibrosis. The present disclosure addresses this unmet need in the art.

### SUMMARY

In some aspects, the present disclosure provides, inter alia, a compounds of Formula (I):

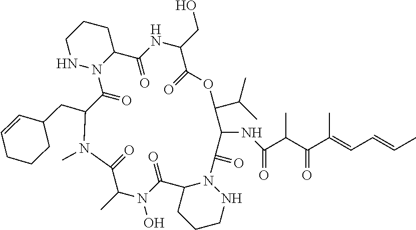 [SMILES: C/C=C/C=C(\C)C(=O)C(C)C(=O)NC1C(=O)N2NCCCC2C(=O)N(O)C(C)C(=O)N(C)C(CC2C=CCCC2)C(=O)N2NCCCC2C(=O)NC(CO)C(=O)OC1C(C)C]

or stereoisomer or mixture of stereoisomers thereof, or a pharmaceutically acceptable salt, solvate, hydrate, or tautomer, thereof.

In some embodiments, the compound of Formula (I) is substantially pure. In some embodiments, the compound of Formula (I) is enantiomerically pure.

In some embodiments, the disclosure provides for pharmaceutical compositions comprising a therapeutically effective amount of the compound of Formula (I) and one or more pharmaceutically acceptable excipients. In some embodiments, the pharmaceutical composition comprises a pharmaceutical carrier.

In some embodiments, the compound of Formula (I) is produced by a host cell comprising a heterologous biosynthetic gene cluster comprising at least six nonribosomal peptide synthetase (NRPS) modules and at least four polyketide synthase (PKS) modules, a set of modifying enzymes, precursor biosynthesis enzymes, transporters, and one or more transcriptional regulators.

In some embodiments, the biosynthetic gene cluster is isolated or derived from Streptomyces griseochromogenes.

In some embodiments, the biosynthetic gene cluster comprises a sequence of SEQ ID NO:1.

In some embodiments, the biosynthetic gene cluster comprises one or more modifications of SEQ ID NO: 1.

In some embodiments, the modification comprises a substitution, deletion, inversion, or insertion of one or more nucleotides relative to SEQ ID NO: 1.

In some embodiments, the modification comprises insertion of at least one promoter sequence.

In some embodiments, the promoter is selected from the group consisting of ermE, kasO, gapdh, and rpslp, or functional variants or derivatives thereof.

In some embodiments, the modification increases synthesis of the compound of Formula (I) compared to an otherwise equivalent host cell comprising an unmodified biosynthetic gene cluster.

In some embodiments, the host cell is a Streptomyces albus cell.

In some embodiments, the host cell further comprises a sequence encoding a Streptomyces Antibiotic Regulatory Protein (SARP) operably linked to a constitutive promoter.

In some embodiments, the present disclosure provides a polynucleotide comprising a biosynthetic gene cluster, wherein the biosynthetic gene cluster comprises one or more genes that contribute to the production of at least a portion of the compound of Formula (I) when the biosynthetic gene cluster is expressed by a host cell.

In some embodiments, the one or more genes comprise six nonribosomal peptide synthetase (NRPS) modules.

In some embodiments, the six NRPS modules are encoded by sequences comprising a first NRPS open reading frame of SEQ ID NO:14, a second NRPS open reading frame of SEQ ID NO: 15, a third NRPS open reading frame of SEQ ID NO: 16 and a fourth NRPS open reading frame of SEQ ID NO: 17, or sequences having at least 90%, at least 95%, at least 97% or at least 99% identity thereto.

In some embodiments, the one or more genes comprise four polyketide synthase (PKS) modules.

In some embodiments, the four PKS modules are encoded by sequences comprising a first PKS open reading frame of SEQ ID NO: 26 and a second PKS open reading frame of SEQ ID NO: 27, or sequences having at least 90%, at least 95%, at least 97% or at least 99% identity thereto.

In some embodiments, the biosynthetic gene complex comprises a SARP-encoding gene.

In some embodiments, the SARP-encoding gene comprises a sequence of SEQ ID NO: 28, or a sequence having at least 90%, at least 95%, at least 97% or at least 99% identity thereto.

In some embodiments, the biosynthetic gene cluster comprises a sequence of SEQ ID NO: 1, or a sequence having at least 90%, at least 95%, at least 97% or at least 99% identity thereto.

In some embodiments, the host cell is engineered to express the one or more genes in the biosynthetic cluster, which results in the production of the compound of Formula (I).

In some embodiments, overexpression of one or more genes in the biosynthetic cluster by the host cell increases the production of the compound of Formula (I) compared to an otherwise equivalent host cell comprising a biosynthetic gene cluster that does not overexpress one or more genes in the biosynthetic cluster.

In some embodiments, the SARP is overexpressed.

In some embodiments, overexpression of the SARP occurs in cis or in trans.

In some embodiments, trans overexpression of the SARP comprises expressing a sequence encoding the SARP open reading frame under the control of a constitutive ermE promoter, or a functional variant or derivative thereof.

In some embodiments, the ermk promoter comprises a sequence of SEQ ID NO:33.

In some embodiments, the biosynthetic gene cluster comprises one or more sequence modifications relative to a biosynthetic gene cluster of SEQ ID NO :1, or a sequence having at least 95%, at least 97% or at least 99% identity thereto.

In some embodiments, the one or more modifications of the biosynthetic gene cluster comprises a substitution, deletion, inversion, or insertion of one or more nucleotides relative to SEQ ID NO: 1.

In some embodiments, the one or more modifications comprise modifications of a promoter of a gene in the biosynthetic gene cluster.

In some embodiments, the one or more modifications comprise insertion of at least one heterologous promoter in the biosynthetic gene cluster.

In some embodiments, the at least one heterologous promoter is a strong promoter.

In some embodiments, the at least one heterologous promoter is selected from the group consisting of ermk, kasO, gapdh, and rpslp, or functional variants or derivatives thereof.

In some embodiments, the sequence of the ermE promoter comprises SEQ ID NO: 33, the sequence of the kasO promoter comprises SEQ ID NO:34, the sequence of the gapdh promoter comprises SEQ ID NO:35, and the sequence of the rpslp promoter comprises SEQ ID NO:36, or sequences having at least 90%, at least 95%, at least 97% or at least 99% identity thereto.

In some embodiments, inserting the at least one heterologous promoter into the biosynthetic gene cluster comprises a nucleic acid guided endonuclease.

In some embodiments, the nucleic acid guided endonuclease is in a complex with at least one guide nucleic acid (gNA).

In some embodiments, the nucleic acid guided endonuclease is a CRISPR/Cas endonuclease.

In some embodiments, the CRISPR/Cas endonuclease is Cas9.

In some embodiments, inserting the at least one heterologous promoter into the biosynthetic gene cluster further comprises a donor template comprising a sequence of the heterologous promoter.

In some embodiments, the biosynthetic gene cluster comprises an mbtH gene upstream of the four NRPS open reading frames, and wherein the at least one heterologous promoter is inserted upstream of the mbtH gene.

In some embodiments, the at least one heterologous promoter is a KasO promoter.

In some embodiments, the targeting sequence of the at least one gNA comprises SEQ ID NOS: 40-44, or a sequence having at least 80%, at least 85%, at least 90%, or at least 95% thereto.

In some embodiments, the biosynthetic gene cluster comprises a sequence of SEQ ID NO: SEQ ID NO: 49.

In some embodiments, the at least one heterologous promoter is inserted between the sequence of the SARP-encoding gene and the first PKS open reading frame.

In some embodiments, the biosynthetic gene cluster comprises an ornithine monooxygenase gene downstream of the second PKS open reading frame, and wherein the at last one heterologous promoter is inserted downstream of the second PKS open reading frame and upstream of the ornithine monooxygenase gene.

In some embodiments, the at least one modification of the biosynthetic gene cluster comprises a modification that results in overexpression of the SARP-encoding gene in comparison to the expression of the SARP-encoding gene by the biosynthetic gene cluster of SEQ ID NO: 1.

In some embodiments, at least one modification of the biosynthetic gene cluster comprises replacement of at least one promoter in comparison to the biosynthetic gene cluster of SEQ ID NO: 1.

In some embodiments, replacement of the at least one promoter comprises replacement a SARP-encoding gene promoter.

In some embodiments, the SARP-encoding gene promoter is replaced with a promoter selected from the group consisting of ermE, kasO, gapdh, and rpslp.

In some embodiments, the biosynthetic gene cluster comprises a sequence of SEQ ID NO: 50.

In some embodiments, the biosynthetic gene cluster is isolated or derived from Streptomyces griseochromogenes.

In some embodiments, the biosynthetic gene cluster produces the compound of Formula (I) in the host cell.

In some embodiments, the present disclosure provides a vector comprising the polynucleotide as described herein.

In some embodiments, the vector is a bacterial artificial chromosomal vector.

In some embodiments, the vector further comprises at least one promoter.

In some embodiments, the vector is suitable for expression in a Streptomyces species cell.

In some embodiments, the present disclosure provides a host cell comprising the polynucleotide as described herein or the vector as described herein.

In some embodiments, the present disclosure provides a host cell, comprising the polynucleotide as described herein and a polynucleotide comprising a sequence encoding a SARP operably linked to a constitutive promoter.

In some embodiments, the constitutive promoter is an ermE promoter.

In some embodiments, the SARP is encoded by a sequence of SEQ ID NO: 28.

In some embodiments, the host cell is a Streptomyces cell.

In some embodiments, the Streptomyces cell is a Streptomyces griseochromogenes, Streptomyces lividans or Streptomyces albus cell.

In one aspect, the present disclosure provides a method of making a polynucleotide comprising a modified biosynthetic gene cluster comprising:
a. providing a first E. coli host cell comprising a first vector comprising a sequence of biosynthetic gene cluster comprising a target sequence;
b. introducing the first vector into a Streptomyces host cell by conjugation;
c. providing a second E. coli host cell comprising a second vector comprising:
   i. a sequence of at least one gNA specific to the target sequence operably linked to a promoter,
   ii. a sequence encoding an endonuclease; and
   iii. a sequence encoding a donor template; and
d. introducing the second vector into a Streptomyces host cell by conjugation; whereby introducing the second vector into the Streptomyces host cell produces a double strand break in the target sequence and introduction of a donor template sequence, thereby generating a Streptomyces host cell comprising a modified biosynthetic gene cluster.

In some embodiments, the biosynthetic gene cluster is an unmodified biosynthetic gene cluster. In some embodiments, the unmodified biosynthetic gene cluster comprises a sequence of SEQ ID NO: 1.

In some embodiments, the endonuclease is a Cas9 endonuclease.

In some embodiments, the donor template comprises, from 5′ to 3′, a sequence homologous to a sequence 5′ of the target sequence, a sequence of a promoter, and sequence homologous to a sequence 3′ of the target sequence.

In some embodiments, the promoter is selected from the group consisting of ermE, kasO, gapdh, and rpslp, or functional variants or derivatives thereof.

In some embodiments, at least one gNA comprises a target sequence selected from the group consisting of SEQ ID NOS: 40-44.

In one aspect, the present disclosure provides a method of making the compound of Formula (I), comprising
a. introducing into a host cell the polynucleotide of the present disclosure or the vector of the present disclosure;
b. culturing the host cell under conditions sufficient for the synthesis of the compound of Formula (I) by the biosynthetic gene cluster; and
c. isolating and purifying the compound of Formula (I).

In some embodiments, the host cell is a an Actinobacterial cell or a Streptomyces cell.

In some embodiments, the Streptomyces cell is a Streptomyces griseochromogenes, Streptomyces albus or Streptomyces lividans cell.

In some embodiments, the host cell comprises a sequence encoding a SARP operably linked to a constitutive promoter.

In some embodiments, the polynucleotide or vector is introduced into the host cell by conjugation with an E. coli comprising the polynucleotide or vector.

In some embodiments, the present disclosure provides a pharmaceutical composition, comprising the compound of Formula (I), and a pharmaceutically acceptable excipient.

In some embodiments, the present disclosure provides a method of treating a disease or disorder in a subject, comprising administering the compound of Formula (I) or pharmaceutical composition thereof.

In some embodiments, the present disclosure provides the compound of Formula (I) or the pharmaceutical composition thereof, for use in treating a disease or disorder in a subject.

In some embodiments, the present disclosure provides a compound of Formula (I) for use in the manufacture of a medicament for treating a disease or disorder in a subject.

In some embodiments, the present disclosure provides the use of a compound of Formula (I) or the pharmaceutical composition thereof, for the treatment of a disease or disorder.

In some embodiments, the disease or disorder is cancer.

In some embodiments, the disease or disorder is fibrosis.

In some embodiments, the subject is human.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the proposed biosynthesis of Formula (I) from the AZT010 biosynthetic gene cluster.

FIG. 2A depicts cloning and overexpression of SARP in trans from the AZT010 biosynthetic gene cluster, followed by detection of the compound of Formula (I) using stable isotopic labeling.

FIG. 2B depicts the target peak of the compound of Formula (I) from an extract of S. griseochromogenes comprising the AZT010 biosynthetic gene cluster, that overexpresses SARP as shown in FIG. 2A. The top left panel (“SG-AZT010-WT”) shows there is no quantifiable peak corresponding to the compound of Formula (I).

FIG. 3 depicts promoter engineering of the AZT010 biosynthetic gene cluster to increase production titer. Promoter insertion sites in the AZT010 biosynthetic gene cluster are shown. The plot at bottom depicts a representative set of titers produced by the SA-pDualP-AZT010-kasO* construct, as compared to other engineered versions of the biosynthetic gene cluster, and the wild type biosynthetic gene cluster.

FIG. 4A depicts the 2D NMR structure of the compound of Formula (I).

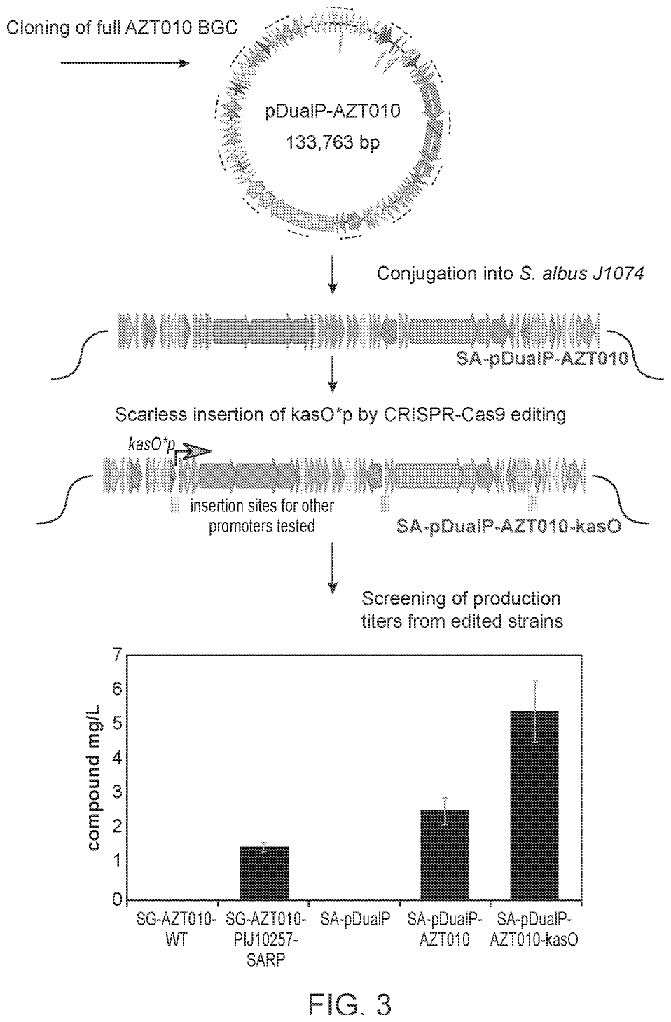

FIG. 4B depicts the HR-ESI-MSMS fragmentation of the compound of Formula (I).

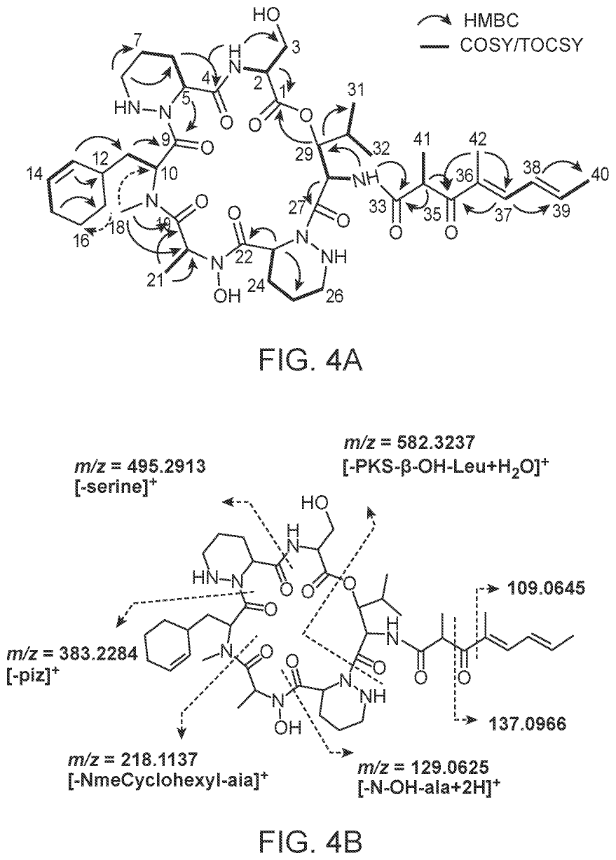

FIG. 4C depicts a mass spectrogram of the compound of Formula (I).

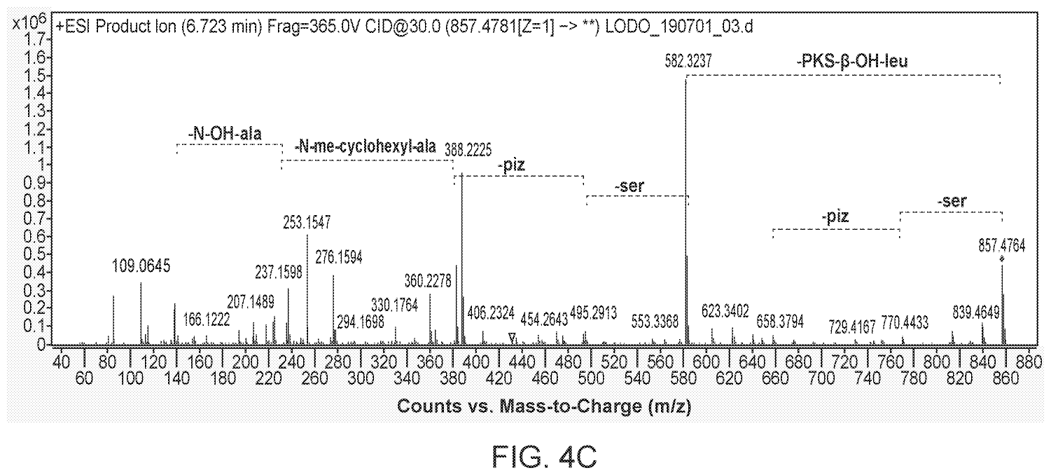

### DETAILED DESCRIPTION

The present disclosure relates to the compound of Formula (I) and to the use of the compound of Formula (I) in the treatment of cancer or fibrosis. In some embodiments, the disclosure relates to the biosynthesis of the compound of Formula (I).

### Definitions

Unless otherwise stated, the following terms used in the specification and claims have the following meanings set out below.

In some embodiments, “the compound of Formula (I)” and “a compound of Formula (I)” includes all stereoisomer, mixture of stereoisomers, pharmaceutically acceptable salts, solvates, or tautomers thereof.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

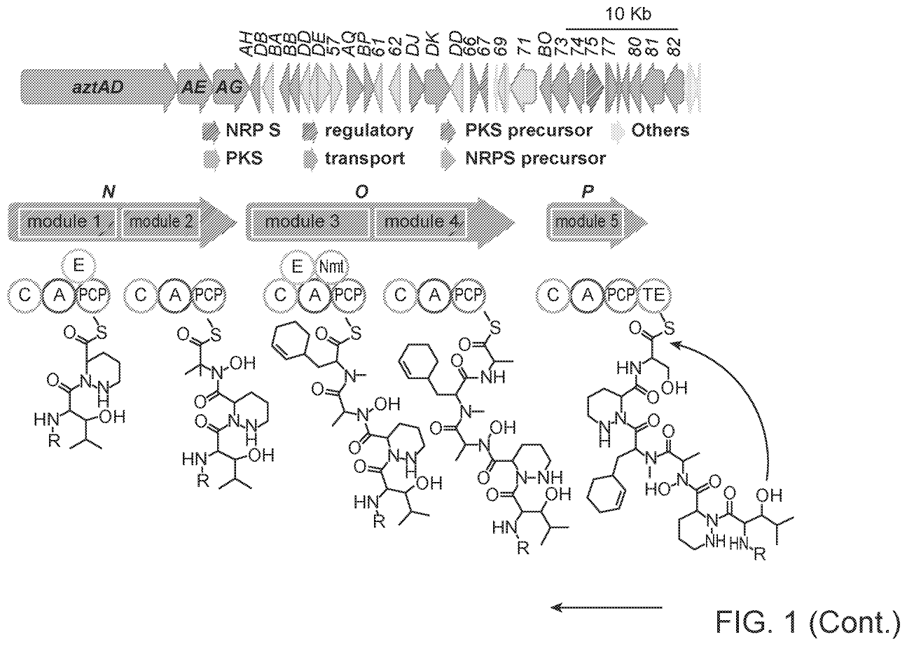

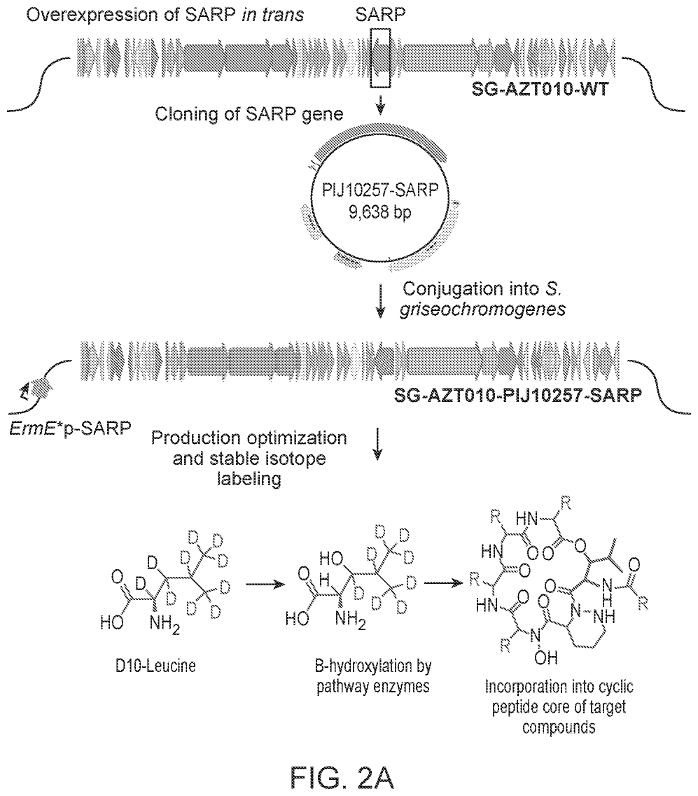

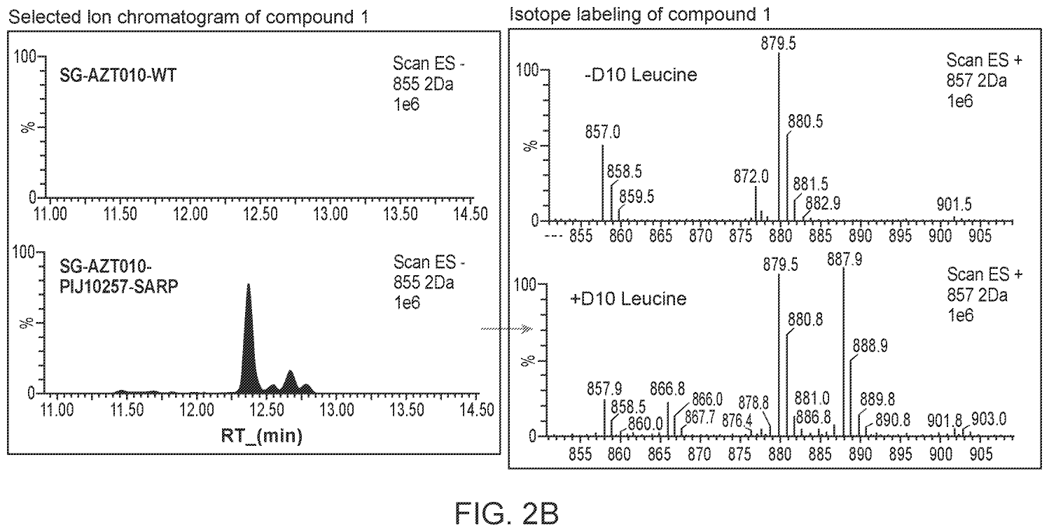

As used herein, “natural product” refers to a compound that is synthesized by a living organism (e.g., bacteria) under normal physiological conditions and the compound can be quantified, and identified as a pathway specific product, using known techniques in the art. If one skilled in the art cannot quantify a compound in, e.g., extracts of native bacterial cells containing a biosynthetic gene cluster after culturing said cells with a growth media containing the nutrients required to produce a compound, one skilled in the art would reasonably believe that the bacterial cells do not naturally produce the compound (i.e., the compound is not a “natural product”). For the avoidance of doubt, the compounds disclosed herein are not “natural products,” because Applicant was not able to quantify the compound of Formula 1 in the native bacterial cells containing the biosynthetic gene cluster. For example, as described in Example 2, Applicant was not able to quantify the compound of Formula (I) in extracts of AZT010 despite culturing said cells under appropriate conditions. (See also FIG. 2B, left panel, compare top (“SG-AZT010-WT”) and bottom (“SG-AZT010-PIJ10257-SARP”); and FIG. 3, bottom panel showing no quantifiable amount of the compound of Formula 1 in wild type (SG-AZT010-WT)).

As used herein, “module” refers to a set of active site domains of a protein that catalyze one or more of the biosynthetic steps leading to the compound of Formula (I). Thus, each module may be composed of a protein, or a module may be composed of a plurality of domains. In some embodiments, heterologous protein domains may be fused together to form a module. In some embodiments, an open reading frame may be polycistronic, and encode a plurality of distinct proteins, each of which comprise one or more domains or modules. In some embodiments, an open reading frame may encode a single protein, which has a plurality of modules, each of which comprises a combination of domains.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

As used herein, the term “treating” or “treat” describes the management and care of a patient for the purpose of combating a disease, condition, or disorder and includes the administration of the compound of Formula (I) to alleviate the symptoms or complications of a disease, condition or disorder, to eliminate the disease, condition or disorder, or to prevent the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “therapeutically effective amount” refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

The terms “polynucleotide” and “nucleic acid” are used interchangeably herein and refer to a polymeric form of nucleotides of any length, i.e., ribonucleotides or deoxyribonucleotides or analogs thereof. These terms refer to the primary structure of the molecule and thus encompass double-and single-stranded DNA as well as double- and single-stranded RNA. The term also encompasses modified nucleic acids, such as methylated and/or capped nucleic acids, nucleic acids containing modified bases, backbone modifications, and the like.

As used herein, the term “gene” refers to any segment of DNA associated with a biological function. Thus, a gene includes, but is not limited to, coding sequences and/or regulatory sequences required for its expression. Genes may also comprise non-expressed DNA segments, e.g. forming recognition sequences for other proteins. Genes can be obtained from a variety of sources, including cloning from a source of interest or synthesis from known or predicted sequence information, and can comprise sequences designed to have desired parameters.

In some embodiments, the genomic DNA, prior to modification, is isolated from bacteria cells originally found in soil.

As used herein, the term “homologous” or “homolog” or “ortholog” is known in the art and refers to related sequences that share a common ancestor or family member and are determined based on the degree of sequence identity. The terms “substantially similar” and “substantially corresponding” are used interchangeably herein. The term refers to nucleic acid fragments wherein the difference in one or more nucleotide bases does not affect the ability of the nucleic acid fragment to mediate gene expression or produce a certain phenotype. These terms also refer to modifications of the nucleic acid fragments of the disclosure, such as deletions or insertions of one or more nucleotides that do not substantially alter the functional properties of the resulting nucleic acid fragment relative to the original, unmodified fragment. Thus, as will be understood by those skilled in the art, it is to be understood that the present disclosure encompasses more than the specific exemplary sequences. These terms “homologous” or “homolog” or “ortholog” or “substantially similar” or “substantially corresponding” may describe the relationship between a gene found in one species, subspecies, variety, or strain and the corresponding or equivalent gene in another species, subspecies, variety, or strain.

As used herein, the terms “endogenous” and “native” refer to naturally occurring copies of a gene or promoter.

As used herein, the term “naturally occurring” refers to a gene that is derived from a naturally occurring source. In some aspects, a naturally occurring gene refers to a gene that is a wild-type (non-transgenic) gene, whether located in an endogenous environment within its source organism or placed in a “heterologous” environment when introduced into a different organism. Thus, for purposes of this disclosure, a “non-naturally occurring” gene is one that has been mutated or otherwise modified or synthesized to have a sequence that differs from a known native gene. In some aspects, the modification may be at the protein level (e.g., amino acid substitution). In other aspects, the modification can be at the DNA level without any effect on the protein sequence (e.g., codon optimization).

For the purposes of this disclosure, homologous sequences are compared. “Homologous sequences” or “homologs” or “orthologs” are believed, believed or known to be functionally related. The functional relationships may be indicated in any of a number of ways, including but not limited to: (a) the degree of sequence identity and/or (b) the same or similar biological function. Preferably, both (a) and (b) are indicated. Homology can be determined using default parameters using software programs readily available in the art, such as NCBI BLAST (basic local alignment search tool).

Percentage identity determinations can be performed for nucleic acids using BLASTN or standard nucleotide BLAST using default settings (Match/Mismatch scores 1, −2) Gap costs linear, Expect threshold 10, Word size 28, and match matches in a query range 0) and for proteins using BLAST using default settings (Expect threshold 10, Word size 3, Max matches in a query range 0, Matrix Blosum62, Gap costs Existence 11, extension 1 and conditional compositional score matrix adjustment).

As used herein, the term “nucleotide change” refers to, for example, a nucleotide substitution, deletion, and/or insertion, as is well known in the art. For example, mutations contain alterations that produce silent substitutions, additions or deletions without altering the properties or activity of the encoded protein or the manner in which the protein is made.

As used herein, the term “heterologous” refers to an amino acid or nucleic acid sequence (e.g., a gene or promoter) that is not naturally occurring in a particular organism or is not naturally occurring in a particular context (e.g., a genomic or plasmid location) in a particular organism. For example, a native promoter or other nucleic acid sequence of Streptomyces albus may be heterologous when operably linked to a nucleic acid sequence which is not operably linked in the wild-type Streptomyces albus, or when the native promoter or other nucleic acid sequence is delivered in a non-native form (e.g.as a heterologous plasmid or heterologous nucleic acid sequence).

As used herein, the term “exogenous” is used interchangeably with the term

“heterologous” and refers to material from a source other than its natural source. For example, the term “exogenous protein” or “exogenous gene” refers to a protein or gene that is derived from a non-natural source or location and that has been artificially supplied to a biological system.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Compounds of the Present Disclosure

In some aspects, the present disclosure provides, inter alia, a compounds of Formula (I):

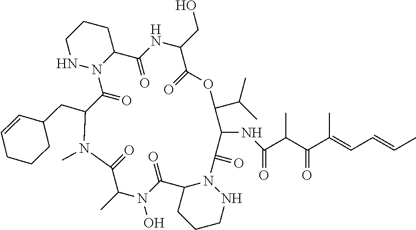 [SMILES: C/C=C/C=C(\C)C(=O)C(C)C(=O)NC1C(=O)N2NCCCC2C(=O)N(O)C(C)C(=O)N(C)C(CC2C=CCCC2)C(=O)N2NCCCC2C(=O)NC(CO)C(=O)OC1C(C)C]

or a stereoisomer, mixture of stereoisomers, a pharmaceutically acceptable salt, solvate, or tautomer thereof.

In some embodiments, the compound of Formula (I) is a stereoisomer, mixture of stereoisomers, a pharmaceutically acceptable salt, solvate, or tautomer of Formula (I).

In some embodiments, the compound of Formula (I) is a stereoisomer of Formula (I).

In some embodiments, the compound of Formula (I) is a mixture of stereoisomers of Formula (I).

In some embodiments, the compound of Formula (I) is a pharmaceutically acceptable salt of Formula (I).

In some embodiments, the compound of Formula (I) is a solvate of Formula (I).

In some embodiments, the compound of Formula (I) is a tautomer of Formula (I).

The compounds of the disclosure are cyclic peptides. The compounds contain a hexadepsipeptide core, which is composed of 6 amino acid cyclized through peptide bonds, and a polyketide tail.

The compounds of Formula (I) comprise nine stereocenters.

In some embodiments, the compound of Formula (I) is Formula IA, wherein each stereocenters is identified with an *:

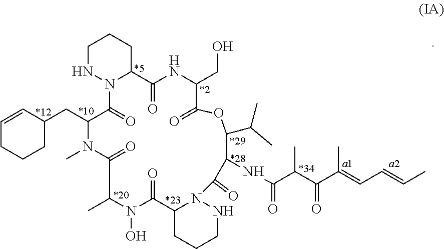 [SMILES: C/C=C/C=C(\C)C(=O)C(C)C(=O)NC1C(=O)N2NCCCC2C(=O)N(O)C(C)C(=O)N(C)C(CC2C=CCCC2)C(=O)N2NCCCC2C(=O)NC(CO)C(=O)OC1C(C)C]

In some embodiments, each * of Formula IA represents a bond which is either (R) or (S).

In some embodiments, *2 of Formula IA is (R). In some embodiments, *2 of Formula IA is (S).

In some embodiments, *5 of Formula IA is (R). In some embodiments, *5 of Formula IA is (S).

In some embodiments, *10 of Formula IA is (R). In some embodiments, *10 of Formula IA is (S).

In some embodiments, *12 of Formula IA is (R). In some embodiments, *12 of Formula IA is (S).

In some embodiments, *20 of Formula IA is (R). In some embodiments, *20 of Formula IA is (S).

In some embodiments, *23 of Formula IA is (R). In some embodiments, *23 of Formula IA is (S).

In some embodiments, *28 of Formula IA is (R). In some embodiments, *28 of Formula IA is (S).

In some embodiments, *29 of Formula IA is (R). In some embodiments, *29 of Formula IA is (S).

In some embodiments, *34 of Formula IA is (R). In some embodiments, *34 of Formula IA is (S).

In some embodiments of the compound of Formula IA, a1 and a2 represent the stereochemistry of the alkene bond.

In some embodiments of the compound of Formula IA, alkene bond a1 is cis. In some embodiments of the compound of Formula IA, alkene bond a1 is trans.

In some embodiments of the compound of Formula IA, alkene bond a2 is cis. In some embodiments of the compound of Formula IA, alkene bond a2 is trans.

In some embodiments, the compounds of the disclosure provide a scaffold which can be derivatized to create therapeutic agents. In some embodiments, the compounds of the disclosure, themselves, are therapeutic agents.

For example, in some embodiments, the compounds of the disclosure target Wnt/betacatenin (or β-catenin) signaling pathway. Wnt/β-catenin signaling, a highly conserved pathway through evolution, regulates key cellular functions including proliferation, differentiation, migration, genetic stability, apoptosis, and stem cell renewal. The Wnt pathway mediates biological processes but the effect depends on the involvement of β-catenin in signal transduction. β-catenin is a core component of the cadherin protein complex, whose stabilization is essential for the activation of Wnt/β-catenin signaling.

Without wishing to be bound by theory, it is thought that the azinothricin family of compounds targets Wnt/β-catenin signaling, and exhibits strong antitumor and antibacterial activity. Alternatively, or in addition, HIF-1alpha and v-ATPase have recently been suggested to be the direct targets of this family of molecules Azinothricin compounds are cyclic hexadepsipeptides that are characterised by a 19-membered cyclodepsipeptide ring composed of 6 unusual amino acids (hexadepsipeptide) and an acyl side chain connected through an amide bond. The first member of this class, azinothricin, was reported from Streptomyces X-14950. Because of the strong antitumor and antibacterial activity, significant efforts have been made to identify bacterially-produced natural products using the classical culturing approach. However, the discovery of new drug candidates using culturing approaches has been limited.

Instead of culturing, Applicant has mined genomes for biosynthetic gene clusters (BGC) similar to those associated with azinothricin, in order to identify potentially new compound structures.

Using this approach, Applicant found a biosynthetic gene cluster (BGC) termed AZT010, was identified from the genome of Streptomyces griseochromogenes strain ATC(' 14511. The AZT010 BGC had potential to produce hexadepsipeptide; however, as described in Example 2, one skilled in the art would reasonably believe that the AZT010 BGC did not naturally produce the hexadepsipeptide of Formula (I). See also FIG. 2B, left panel, compare top (“SG-AZT010-WT”) and bottom (“SG-AZT010-PIJ10257-SARP”); and FIG. 3, bottom panel showing no quantifiable amount of the compound of Formula 1 in wild type (“SG-AZT010-WT”)).

The BGC in AZT010 contains a Streptomyces Antibiotic Regulatory Protein (SARP) regulator aztT10 in the middle of the core genes. A SARP regulator is a class of BGC specific regulators that act as positive regulators of compound expression in many species of Streptomyces. The inability to quantify compounds of Formula (I) suggested that SARP expression levels were not sufficient to produce the compounds of Formula (I).

Thus, Applicant overexpressed SARP aztT10 by cloning it into an integrative plasmid, PIJ10257, under the control of constitutive ermE* promoter—a strong heterologous promoter not present in the naturally occurring AZT010 BGC. The promoter was added to initiate transcription of RNA, and consequently synthesize the compounds of Formula (I).

### Polynucleotides and Vectors

The disclosure provides polynucleotides comprising a biosynthetic gene cluster comprising one or more genes that contribute to the production of at least a portion of the compound of Formula (I) when the biosynthetic gene cluster is expressed by a host cell. Host cells expressing the polynucleotides of the disclosure can be used in the manufacture of the compound of Formula (I). In some embodiments, the biosynthetic gene cluster (BGC) can be wild type, i.e. not subject to modifications through genetic engineering methods known in the art. In other embodiments, the BGC is subject to one or more modifications, for example modifications that increase, or result in, expression of the compound of Formula (I) by the host cell.

### Biosynthetic Gene Cluster

In some embodiments, the biosynthetic gene cluster involved in the production of the compound of Formula (I) is isolated or derived from a Streptomyces species of bacteria. Streptomyces are a species of Actinobacteria, and the type genus of the family Streptomycetaceae. Over 500 species of Streptomyces have been described to date, all of which are envisaged as within the scope of the instant disclosure. In some embodiments, the biosynthetic gene cluster is isolated or derived from Streptomyces griseochromogenes.

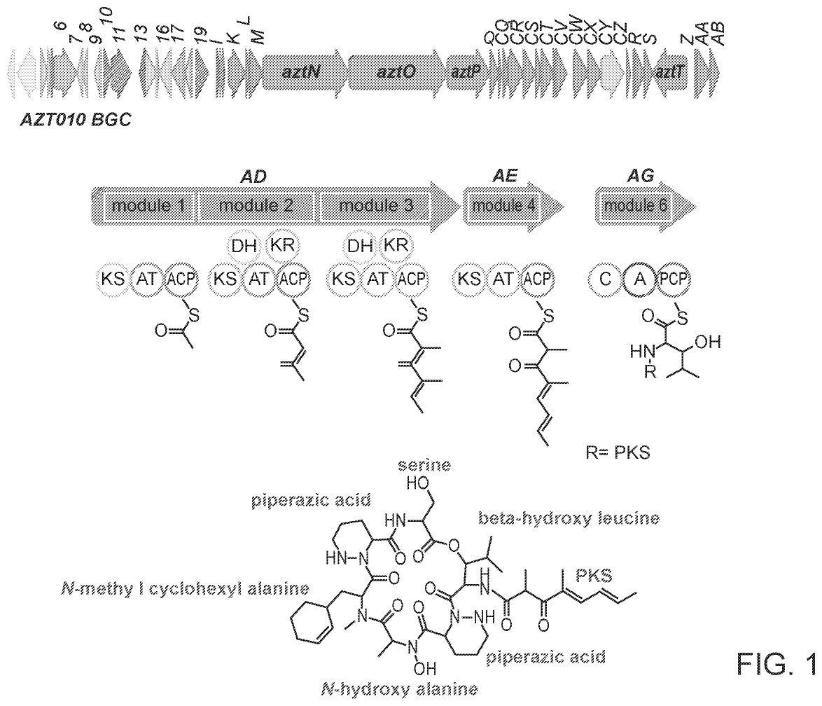

In some embodiments, the biosynthetic gene cluster comprises one or more genes that contribute to the production of at least a portion of the compound of Formula (I) when the biosynthetic gene cluster is expressed by a host cell. As a non-limiting example, the biosynthetic gene cluster comprises at least one gene that, together with other genes in the host genome and/or the biosynthetic gene cluster, catalyzes or contributes to at least one biosynthetic step that results in the production of the compound of Formula (I) from a precursor compound. Exemplary precursor compounds are shown in FIG. 1.

In some embodiments, the biosynthetic gene cluster comprises at least one nonribosomal peptide synthetase module. Nonribosomal peptide synthetases (NRPS) are enzymes which, unlike ribosomes, synthesize their own peptidic products independent of messenger RNA. NRPS are modular enzymes that catalyze synthesis of important peptide products from a variety of standard and non-proteinogenic amino acid substrates. Typically, each NRPS can synthesize one type of non-ribosomal peptide. Nonribosomal peptides often have cyclic and/or branched structures, can contain non-proteinogenic amino acids including D-amino acids, carry modifications like N-methyl and N-formyl groups, or are glycosylated, acylated, halogenated, or hydroxylated. The NRPS genes for an individual peptide are frequently organized into operons in bacteria. Functionally related operons may be organized into gene clusters. NRPS enzymes are organized in modules, each module comprising multiple catalytic domains that are responsible for incorporation of a single amino acid residue. In an exemplary NRPS module, a first domain activates and covalently attaches an amino acid to an integrated carrier protein domain, and the substrates and intermediates are then delivered to neighboring catalytic domains for peptide bond formation or, in some modules, chemical modification. In the final module, the peptide is delivered to a terminal thioesterase domain that catalyzes release of the peptide product. All NRPS, and modules thereof, that are capable of contributing to the production of the compound of Formula (I) are envisaged as within the scope of the instant disclosure.

In some embodiments, the one or more genes comprise at least one nonribosomal peptide synthetase (NRPS) module. In some embodiments, the one or more genes comprise at least 1, 2, 3, 4, 5, 6, or more NRPS modules. In some embodiments, the one or more genes comprise six NRPS modules. Sequences of representative NRPS modules are described in Table 1, below.

TABLE 1
NRPS Modules

| | | DNA |
|---|---|---|
| Name | Amino Acid Sequence | Sequence |
| aztN10 | IPLSFAQRRLWFVDRFEGPSPAYNGPLALRLTGELNVGALQAAVGDLIDRHEALR | SEQ ID |
| (module | TVIVEDDDGVPHQRVLPSGQKRFGLRVVEAATEEERAAAVDEAATGTFDLAADVP | NO: 2 |
| 1) | IRARLVRRGPREHTLVLVMHHIAVDGESFGPLCRDLITAYTARQEGRAPAWEPLP | |
| | VQYADYTLWQRDVLGDEADPHSLAARQLEYWRRTLADLAQPLAFPTDRPRPKTMS | |
| | HHGDMVPFSIDPGLLRSVEKLAAQADTTVSMVMQSALAVLLHHLGCGDDVPIGAP | |
| | IAGRTDEELRELVGFFVNTWVLRVDLSGNPTFRELLQRVRERALAAYDNQDMPFE | |
| | RLVELLNPDRSTAYNPLFQVMLAWQPPIPEPEFPGLDVQSERLETATAKFDLFFD | |
| | LIPHGSGGARCRLEYRTDLFDRDTVEGIANRFVRVLGRLVADAERRIGGIDVLDA | |
| | AERGRLLTQFNDTATAVPELTVPELFESQVARTPDAPAVVCDDRTLTYRELDERA | |
| | DGVARELVRRGAGPEDLVVLALPRTEDLVAGLLGILKSGAGYLPMDPQYLAGRAE | |
| | SVLAEARPRFVVTDTKTSQDLPPNDMSSIYLDDRAQWDAPVKTPDDGGRISPPRP | |
| | DNLAYVMYTSGSTGKPKGAAITHRNVVNGVRELSRVLDAPPGWRMLAGTSVNFDV | |
| | SVFELLTTLSTGGTAEVVPNALALGERDSWDGHVISAVPSVLGELVGHLEKTTDV | |
| | RTVVFAGDVLPARLVRQVREALPGVQVVNSYGQSESFYATTFALPASEEWAESEV | |
| | APIGTPLGNMRAYVLGPGLAPVPQGAVGELYVVGTCLGRGYHGRPGLTAERYVAN | |
| | PFGPAGERMYRTGDLARWNARGRLECLGRGDGQVKVRGFRIETAEVEAALTAHPG | |
| | ISEAVVISRDVSPGGRRLVAYVVHAGEGAVGDDGAGGIGDVDVLAGASAAELRTF | |
| | VAARLPDYMVPSAFVALDRMPLGPTGKLDRSALPEPEFVGEGYREPRTEAEAIIT | |
| | VAYADVLGVERVGIDDDFFAVGGDSLRSIQVVARARARGLDLTTREIFECRTAAR | |
| | LAEVASARQDRAPALAELEGGGVGPMPLQPVARQVFEHGGGMNRFAMSMVLELPA | |
| | GIDERGLAATLDAVLDRHDLLRARLVRGDEFSLVAQPQGSVRAADLIRRVGCDGR | |
| | WDDPSSLEAAKAELDDAVGRLDPEAGTMADFVWFAPEAGTGRLLVVLHHLVVDGV | |
| | SWRILMSDFAEAWQQVRAGRTPELPAVGTSARRWASALEDEALSAEREAELAYWR | |
| | DLLEVPDPALGTRVFDPAVDVMSTVDTVRVQVPADVTEAVLTRLPAAFKGTGTDV | |
| | LLAALALAVNRWRGADRSALVRLEGHGREEDVVPGADLSRTVGWFTSMYPVRVDV | |
| | (SEQ ID NO: 8) | |
| aztN10 | EVWPQAPGQSGIQFEAALADGSFDVYHMQFVLHLSGHVDPERMRSAGQALLDRYP | SEQ ID |
| (module | NLRSAFLAAAGGDPVQVVADHVALPWRHIDLTGRAAAEQDAALDQALADDRADRL | NO: 3 |
| 2) | DPGKPPLMRLALLTCGPRQAKLIITAHHTLFDGWSSPLVIKDLIRLYSTAGGLGP | |
| | VRGYGDYLTWLSTRDRKASAAQWAAELTGFDQPTLVAPNAPVQEAASALGRVEVP | |
| | LSIDKGRELARRAAELGVTLNTLLQGAWGILLSKLTGRQDVVEGAAVNGRPAELA | |
| | GSDEMVGLFINTLPTRASCRPDHSVAQVITDLQNRQTALLDHHYYGLADIQRDVG | |
| | LPALFDTIVVFENYPIDRAGIVDANTAAGFTIDAIRPFAGSHYPLTLNASDPYLR | |
| | TSLDYQNNLYDREAAELIAARLVRVLDQVLDDPTVPVGAVEVLSREEWDRLVRRV | |
| | NDTARPVAADTLPGAFEAQVARDPDRVALIGERERLTYGEFDRRANQLAHWLVEQ | |
| | GAGPEQLVAVRIPRSVDLMVAIYAVVKAGAAYVPLDTELPEDRVRHVLDSAEPLL | |
| | VLDGTLPDVSAYPTTNPERVLSPDNAAYVIFTSGSTGGPKGVQVSHRSIMNRLKW | |
| | GLAHFDVGTEDRVLLSTSASFDVSVPELFAPLQVGAAVVIARPDGRRDPSYLAEL | |
| | IRRERVTGADFVPSLLEAFAGEPAAKRCDSLRWIEVAGEAFPAALANKVVDLLPD | |
| | CGVHNLYGPTEAAVEVTAWQHVPGADRVPIGAPVWNTQVYVLDAALRPVAPGVAG | |
| | ELYLAGAGLARGYLGRSALTANRFVACPFGPAGARMYRTGDLVRWNEDGQVEYIG | |
| | RTDFQVKVRGFRIELSEVESALTAHPDVDNAVVVVREDRADDQRLVAYVVPGGGR | |
| | ADPSGLDLAALTDLVRGRLPEYMVPSAIVPLAAFPTTASGKLDRKALPAPDHTEA | |
| | TAGRGPRNHHEEVLCRLFAELLGVEEIGIDVDFFDHGGHSLLATRLIGRIRSEFD | |
| | ADVKVTTVFRHPTVAQLAEQIQK (SEQ ID NO: 9) | |
| Azt010 | IPLSFAQSRMWILHKLEGPSATYNVPFVLRLEGVLDTTALATAVTDVTNRHESLR | SEQ ID |
| (module | TLVVEDAGGTASQRIVTPEEAVFPFRVVDVAADAVDAAMHEVACEPFDLDTELPL | NO: 4 |
| 3) | RTTVLRIAPQEHVLVFVFHHIAADGASVAPFVRDLVSAYTARHRGSAPQWTPLPV | |
| | QYKDYTLWQRQLLGDETDPESTAAEQIAYWKKELAEVPQPLQLPLDRPRPTAASH | |
| | RGGEVPFVLAPELLSGVQKLAADHGATAPMVAQAALAVLLHKLGAGEDVPIGSPI | |
| | EGRGDEQLDDLIGFFVNTWVLRADLSRNPSFADLLEQVRDKALAAYDNQDIPFER | |
| | LVELLNPDRTTAYMPLFQTALGWQFVWGEIEMPGLRVTPIPVGTGTAKFDLLENI | |
| | VPNASGGTRGLLEYATDLFDHTTAERIVDRFVRILEQVLEDPAVPVGAIEVLSAQ | |
| | EQDWLLRGATDTALSVPERTVDALFSERAAATPDAVALVCGDVTLTYREVDERAN | |
| | RLARVLTERGVEPESVVAVVVSRSPQWVVALLGVMKAGGAYLPVDPAYPAERVAF | |
| | MVADSGAVLVIGDAVSAGQVPELSAPMIRLDDPDVVAALAGADPGPVTDADRRGP | |
| | VAVANTAYVIYTSGSTGTPKGVAVSHSGVASLAASQAERLAVTPDSRVLQFASPS | |
| | FDASLWEWSMALLTGAALVLAGPDELAAGDPLIETIAAHGVTHATLPPVVLAALP | |
| | TGALPSVETLVVGGSASSPELVAQWSAGRRMINGYGPTEITVCAAMSTAMVGDGR | |
| | TPPIGRPIANTQAYVLDTALRPVAPGVAGELYIAGPGLARGYLGRTGLTADREVA | |
| | CPFGAPGTRMYRSGDLVRWNQDGELEYLGRTDFQVKIRGFRIELGEIEHALTMHP | |
| | GVAQAAVVVNENQLGDKQLVGYLVPKPYAAAVAGADAQVDEWRHLYDDSYADSSD | |
| | EELGEDFQGWNSSYTGEPIPREQMLEWQDAAVAQVLRFEPRRVLEIGVGSGLLLA | |
| | KIVDEVEEYWGTDISATVVDRVRAQAAEAGYGDRVRVSAQPADDMSGLPRGRFDT | |
| | VVLNSVVQYFPSVEYLDQVLRQVMELLVAGGRVIVGDVRNAATRTLMQTAVQRTA | |
| | HPHASHDELRTLVKRELLAERELAIAPEWFTEWAGDDSVAVDIRLKPGQAHNELT | |
| | RHRYEVVLYKRPADVLDLAGVPSVPWGREVHDLADLGRCADRAGGPVRVTGIPNA | |
| | RLAEEAALTVATGLLDPAPRSGEPVDPQELLQWARKDDRDVVLTWSGEDTRCFDA | |
| | VLLPERRTGQPHVSGSFVPSGAGGRVRANNPALARSIGPLLIELPEYLRERLPDY | |
| | MVPPALVPLSELPLTPNGKLNHRALPAPDYGQAATGRAPRNQVEETLSALFAEVL | |
| | GVDNVGIDNDFFATGGDSIRSIQIVARARARGIEVSTREIFEHRTVARLADLVEG | |
| | REEERLTLAELPGGGVGWAPLMPTAKHVLALGGGLGRLCMSMMLTLPEGIDRAGL | |
| | VATVQTVLDRHDVLRSRLDRTRQGLSIEPAGSVDAGALLREVRYGDADAHAELDA | |
| | AADRLDPDAGVMAQFVWFTSDTDTDADRLLIVLHHLVVDGVSWRLLLPDLVSAWK | |
| | QVRDGRTPEPAGPGTSLRRWAHALADEAARPERVAELPVWRQILRGDEPVLGARE | |
| | LDPARDVAATVETVRVHVPADVTETVLTKVPAVFRGGVDDGLLAALALALTRWRR | |
| | TRGVPASSALVRLEGHGREEEAVPGADLSRTIGWFTTMYPVRLDLA (SEQ ID | |
| | NO: 10) | |
| Azt010 | LAEVWPVTSAQSGILFHSMLAGSSFDAYHVQLVFHISGDVDPERMSRAGQMLLER | SEQ ID |
| (module | HTSLRAAFVDGADGDLVQVVPAAGVTLPWRYLDLTGYGEAERTETFERFLAQDQA | NO: 5 |
| 4) | AHFDVGTPPLIRLALVALEPGRAELVMTVHHSLADGWSSPLLLQDLLLLYASHGD | |
| | AAGLPGTRSYGEFLAWRARQDQDEAARAWAAELDGVDEPTLLAPGATGGDGLDQV | |
| | EVALPLDLSGELNRCASVLGVTINTLVQGAWALLLGQLTGRQDLVFGATVSGRPP | |
| | AVTDVETMVGMFINTLPVRVEYAPGDTLAEVLTRLQSRQAGLVEHHYYGLTEIQQ | |
| | SVGLQNLFDTLVVFESYPVDRDGLSSATDAADGIAITGLRPSNGTHYPLALMAAV | |
| | DTHLQFLLQYTPGVFDRDTVEAYAARFVRILEQLAATPELKTAQLDVLEPAERDR | |
| | LLVEFNDTAVPTPDVSVNALVEAQAARTPDEVAVVAEGESLTYREVNARADRLAC | |
| | ELAARGVGPESMVAVSLPRSADLVVALLAVLKAGGTHLPVDPRYPSHRLAAIFEE | |
| | ARPHLVLTDQATVGVLPEHDAPDVLLGSLDLSGDVTGLEIPSHADQLAYVMYTSG | |
| | STGKPKGVAITHGCVVNAVLRLAPIVGMEPGKRLLAAASVNFDISVFEIFTTLAT | |
| | GGVVDVVQDVLVLGDRKGWSGSVIHTVPSAFAELVDDIADRTSVETLVFAGEALP | |
| | SSLVEKTRDAFPGVRIVNAYGQTESFYATTFTVDGERPVSAGSAPAGAPLGNNRA | |
| | YVLGPGLTPVPPGAVGEIYIGGNVARGYHGRAALTAERFVPDPFGPTGARMYRTG | |
| | DLGRFNGEGQLEYVGRGDAQVKVRGFRVEPAEVEAALSAHPGVAQTAVIARDGRS | |
| | TGKQLVAYVVPAVTGDSTTTGDPRGPETKELHDFVAERLPDFMVPSAFVVLDRLP | |
| | LATNGKLDRAALPEPEYTGAAYRAPRTAREESLAALFAEVLGVDRVGIDDGFFEL | |
| | GGHSLLATRLISRARAEMGIEIPIRKIFDLPTVAALAAWSEE (SEQ ID NO: | |
| | 11) | |
| aztP10 | IPLSFAQRRMWILHQMEGGSENWNMPAAFRLTGALDQAALTAAIRDVVDRHETLR | SEQ ID |
| (module | TVYETNDDGELYQRILPTAEATPEVPVIEVAPGDVSGTIEEAFGYREDLAAEPPL | NO: 6 |
| 5 | QVRLFRCSPQEHVLVLVIHHIATDGSSVAPLVRDLAAAYTARRDGRAPAWEPLPV | |
| | QYKDYTMWQRELLGDVADPDSLAAAQVAYWRKELAGVPQPLNLPLDRPRPVQASG | |
| | RGSTVGLMVEPEVASGLQKLADERGATMSMVLHAALAVLLRKLGGGDDVTIGSSI | |
| | AGRTEEALADLVGFFVNTQVLRVDLCGDPSFTDLLAQVRHKSLAAYEHQDVPFDM | |
| | LVEAINPERSAAYQPLFQVMLGLQNYERPELELAGLTLEVEQTIPSTSKVDLFEN | |
| | MIVDDSGALRGDISYATDVFDRETVEAFGARFVRVLEQLVGDPGMCVGDVDVLSP | |
| | GERQRLVVEVNDTAEPTLELVEAVRRQVRATPEALAVIGEEESLTYQELEARSNR | |
| | LAHWLADLGVRAESRVAVCLPRTVNLVVALLAVIKAGGAYVPIDPEHPRSRIDYI | |
| | LEHADPLLVLDAEALAGVDWPAYPDAAPEVVVRPENAQYVIYTSGSTGKPKGVAI | |
| | PRGAVANYLATTQRRFPLSSADRMLFSTTVAFDMANTELYLPFTAGATMVMAGKD | |
| | TVTDPSAVLELIRRHGVTAVQATPAFWQMLLMHDPNAAKGLRIIIGAEAVPVRLA | |
| | ETLAKQAAEVENMYGPTETVTWCTEARVEVGQGAPIGRPVGNTQVYVLDSRLSPV | |
| | SRGVPGELYIAGAGMARGYQGRPELTAERFVACPFGPAGARMYRTGDLVRWDGNG | |
| | QLEYLERTDFQVKIRGFRIELGEIEHVLTGHPGVAQAAAVVRENQDGDKRIVGYV | |
| | IPEPDASQADGGVQALLAELPAYLRGRLPDYMMPSTLIPLSEVPLTPNGKLDRGA | |
| | LPSEDINAAVSREPRNSHEERMCALFGELLGIERVGIDDDFFELGGHSLLATRLS | |
| | ARIRGEFDIEMPLRTIIKYPTAGELAALLLVTTPAESSDPLAVVLPLNSDPGTGK | |
| | PPLWFFHGGGGLGWTYFSFASYVPDWPAYALQARGSDGVDKLAGSVKEMVEDYVT | |
| | EMLKIQPEGPFHLIGWSYGGTVVQAVAEALDRRGHEIAFVAILDSQPGGHGFTEI | |
| | HAGKALPDYRSELEEFFGQYIGTDNRQDFLDAMAKVLANNHTLMMDFESPVYRGD | |
| | VLFFSATLQERSYAHLWRPYVLGSIEVHDVRATHHEMNMPAAVAEVMEVIKRKL | |
| | (SEQ ID NO: 12) | |
| aztAG10 | MISVRSFGASSAHKGLWRAQEMFPDTLNHALTLWNVDGELDAAVMESAFLHVMGE | SEQ ID |
| (module | AEVLRVTFVDDGDGLRLVPRELGDWRPFFLDTGAEDDPEQAAREALADMVRQPED | NO: 7 |
| 6) | LERDLLFRLGVVRLAAARSVLVIIYHHLISDGFGAAGLLSRRLAEVYTALSRGAD | |
| | VPELPHPWDAESFATEAAQYSASEQFAEDTEFWRDYLADAPAPARIPRVALSDAM | |
| | RSALSEPMGSADRWGELTEPIGMVSRTLTVPHADAQAWTEAAKSMGVWMSSMLTA | |
| | AAAVFLRHRCDRPEFLLSLAVANRVGAASRTPGLAVNVVPVRVKVPLGATFTEIA | |
| | DAIGDETYEIFDHAACHYSEIQRASGTVLNDRGSFGAVVNVVEFAERLRFADSPA | |
| | HFLGGTTGTFEELAIGVYTDGTADSDLFIRLDAPASLYSRAELRFIGEDLIAHIR | |
| | AVVAADEQPVGALDVVSGAERDRVLTAPNDTNVPVPGLRVPELFARQVDRAPDAV | |
| | AVVSGDSAVSYRELDERSSRLAGALRQRQVGPETVVAVALPRSVDLVVALLGVVK | |
| | AGGAYLPVDPTFPAARIAPVARDASVRALLTDMATAEALSGDLDVPAILEDDIRP | |
| | DTAADSDAATEVPLPSRQDGLLAVMYGSGPTGAATGVAVTHRNMERFALDRRWRE | |
| | GGRDTVLWHAPHTSDALPLEVWVPLLNGGRVVVAPPGELDIDALTEVRAAHEIST | |
| | VWLPAGLFSAIAAERPERLAGLREVWTGGERVSAAAVRRVREACPELTVVTGHGP | |
| | TETTVFAASHRMAADEPVHHAGAVGRPMDHTALYVLGPGLAPVPVGVAGELYVAG | |
| | PGVTRGCPGRPGPTAERFVPCPFGPAGARMYRTGDRVRWSTDGLLEYVGRAGARA | |
| | DVRGTRVELAEVEEALSEHTSLARSVVVVSEDTSGQQRLVAYVVPVGGRTVAGDE | |
| | LRRFAAGRLPEFMVPSVFVVMERLPLTAGGRVDRYSLPEPTFDDDKYRAPRDHTE | |
| | RVLAEAFADVLELDRVGIDDDFFDHGGNSLRAIRLTGLIRAELNQEVPIRTLFAA | |
| | RTIAGLSDSWKELARSSRPTLRRRTKEGEAL (SEQ ID NO: 13) | |

In some embodiments, the biosynthetic gene cluster comprises a sequence encoding one or more NRPS modules. In some embodiments, the sequence encoding the NRPS module is selected from the group consisting of SEQ ID NOS: 2-7, or a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, the sequence encoding the NRPS module comprises, or consisting essentially of, a sequence selected from the group consisting of SEQ ID NOS: 2-7.

In some embodiments, the biosynthetic gene cluster comprises sequences encoding six NRPS modules. In some embodiments, the sequences encoding the six NRPS modules comprise sequences of SEQ ID NOS: 2-7, or sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, the sequences encoding six NRPS modules comprise sequences of SEQ ID NOS: 2-7.

In some embodiments, biosynthetic gene cluster comprises one or NRPS modules comprising a sequence selected from the group consisting of SEQ ID NOS: 8-13, or a sequence having at least 90%, at least 91%, at least 92%, at least 93%, at least 94%, at least 95%, at least 96%, at least 97%, at least 98%, or at least 99% identity thereto. In some embodiments, biosynthetic gene cluster comprises one or NRPS modules comprising a sequence selected from the group consisting of SEQ ID NOS: 8-13. In some embodiments, biosynthetic gene cluster comprises six NRPS modules comprising sequences of SEQ ID NOS: 8-13, or sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, biosynthetic gene cluster comprises six NRPS modules comprising sequences of SEQ ID NOS: 8-13.

In some embodiments, the six NRPS modules are organized in 1, 2, 3, 4, 5 or 6 open reading frames. In some embodiments, the open reading frames comprise sequences selected from the group consisting of SEQ ID NOS: 14-17, or sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, the six NRPS modules are organized in 4 open reading frames. In some embodiments, the six NRPS modules are encoded by sequences comprising a first NRPS open reading frame of SEQ ID NO:14, a second NRPS open reading frame of SEQ ID NO: 15, a third NRPS open reading frame of SEQ ID NO: 16 and a fourth NRPS open reading frame of SEQ ID NO: 17, or sequences having at least 90%, at least 95%, at least 97% or at least 99% identity thereto. In some embodiments, the six NRPS modules are encoded by sequences comprising SEQ ID NOS: 14-17.

In some embodiments, the biosynthetic gene cluster comprises one or more genes that contribute to the production of at least a portion of the compound of Formula (I) when the biosynthetic gene cluster is expressed by a host cell. In some embodiments, the one or more genes comprise at least one polyketide synthase (PKS) module.

In some embodiments, the one or more genes comprise at least one PKS module. In some embodiments, the one or more genes comprise at least 1, 2, 3, 4 or more PKS modules. In some embodiments, the one or more genes comprise four PKS modules. Sequences of representative PKS modules are described in Table 2, below.

TABLE 2
PKS Modules

| | | DNA |
|---|---|---|
| Name | Amino Acid Sequence | Sequence |
| AztAD10 | IAVVGMSCRLPKARNPLAFWELLRDGRSGITDVPADRWDAHRLLAADVT | SEQ ID |
| (module 1) | APGKVTTTRGGFLDDIAGFDADFFGIAPNEAAMMDPQQRLMLELGWEAL | NO: 18 |
| | EDAGIVPEHLAGTRTGVFVGAIWDDYAVRLYKHGTRRIDRHSVTGLHRS | |
| | IIANRLSYTLGLNGPSLAVDAAQSSSLVSVHLAAESLRAGECTLALAGG | |
| | VNLTIVPESTIGSTKFGGLSPDGLCKTFDARANGYVRGEGGAYLALKTL | |
| | SRAVADGDRIYCVIEGSAVNNDGATPGLTVPSARAQEEVIRRAHDRAGT | |
| | RPEDVQYVELHGTGTRVGDPIEAAALGAALGTGRPADAPLHVGSAKTNV | |
| | GHLEGAAGIVGLLKTILSIWHRELPPSLNFETPNPDIAFDELRLRVQQD | |
| | LTSWPRPDRPLIAGTSSFGMGGTNCHVVLREWTVDDCASVQDGPAVVTD | |
| | GTVPWLVSAKSRAALEEQARRLLDHLDRHPRETPAEVGHALAVGRSVED | |
| | HRAVVVGREPADERGALSALAEGEPSAQVVTGAVAARPGKTVFVFPGQG | |
| | SQWVGMGVELMASSPVFAEHLTACAAALEPYTGWNLIDVLAQAQGAPAL | |
| | EGDDVIQPALWAVMISLARLWEHLGITPDAVVGHSQGEIAAAHIAGVLS | |
| | LEDSARIVALRSQALVQIAGHGGMVSLPLPLADASELIGRWEGRLVVAT | |
| | VNGPSATVVAGDTDAVEELRAHCEKEGFRARRVPIDYASHTSHVHPLRD | |
| | RLLDVLAPIEPREASIAFYSTVAGHLGGPMADATVMDARYWYDNLATAV | |
| | HFQAATSALLDDGHTLFIEASPHPVLTHPLQETVEDHASGGEVVVTGTL | |
| | RRDDGTWQRVLTSLATVHTHTIAPVDWSGFFPATRPTHLDLPTYPFQRR | |
| | RHWIDLPTDGVADADSDASVDVVADVVADRPEDTAPPAESGASSPLLDR | |
| | LRKASRTQREQILTDRIRAEVAAITGRVTSETVDGDRTFKDFGFDSFAS | |
| | VELRNRLSALTGLKLPTTLLENHPTPTAVARYLRTEL (SEQ ID NO: | |
| | 22) | |
| AztAD10 | IAIVGMACRYPGDVRAPEDLWRLVSEGVDAITDFPADRGWDTEKLLDPD | SEQ ID |
| (module 2) | PDRPGTSYVTKGGFLSEAAAFDPAFFGIGPREATAMDPQQRLLLETSWE | NO: 19 |
| | AIEQAGIDPAALRGSQTGVFVGAMTQEYGPHLHDGVAGFDGYLLTGNTA | |
| | SVASGRISYTLGLQGPAVTVDTACSSSLVSMHMAAQALRNGECSLALAG | |
| | GVAVMATPGMFVEFSRQRGLAADGRCKAFAEAADGTAWAEGVGLLLLER | |
| | LSDARRNGHQVLAVVRGSAINQDGASNGLTAPNGPSQERVIRQALAGAG | |
| | LSAQEVDVVEAHGTGTALGDPIEAQALIATYGKDRPEDRPLWLGSLKSN | |
| | IGHSQAAAGVGGVIKMVMAMRHGVLPQTLHVDQPSSHVDWAEGAVELLT | |
| | ENRTWPESDHPRRAAISSFGISGTNAHLIVEQAPEGEGAAGEGVPEPVV | |
| | VTDGTVPWLVSAKSRAALEEQARRLLDHLDRHPEATPVEIGHALAASRS | |
| | VFDHRAVLVGREPDDERGALTALAAGEPSARVVAGTAAAQPGRTVFVEP | |
| | GQGSQWAGMGVELMRTSPVFAEHLTACAAALEPHTGWNLVDVLAQEQGA | |
| | PALEGDAVIQPALWAVMISLARLWEHLGITPDAVVGHSQGEIAAAHIAG | |
| | VLSLEDSARIVALRSQALVQIAGRSGMVSLPLPSAGASELIGRWEGRLV | |
| | VATVNGPSATVVAGDIAAVEELLAHCESEGLRARRVPIDYASHTSHVHP | |
| | LREQLLDTLASVEARAATVAFYSTVAGHLGGPMADTTVMDAEYWYDNLA | |
| | TAVHFQAATSALLDDGHTLFIEVSPHPVLTHPLQETAEDHPTGERTLVT | |
| | GTLRRDDGTWQRVLTALATAHTHTTTPVGWSDFFPATRPTHLDLPTYPF | |
| | QHQRYWLERPESAGDAAAAGQVGVEHPLLGAAVELAGTDVTVLTGRLSL | |
| | QTHGWLADHTISGTVLLPGTAFVEMALRAGDEVGVDHLEELALQAPLVL | |
| | PATGGVQLRVEAGELDDTGRRSVSVYSRPDGQGTGVPWTCHATGVLATS | |
| | GPAPSSWDPRVWPPAGAVPVETDELYPSLAVLGYQYGPAFQGVRTVWKR | |
| | GDEVYAEVVLPQERHAEVAAFGIHPALLDAALHAGLVPDPVAGWEPEPP | |
| | RLPFVWSDVRLHATDATNVRVRLAPAGHDALVLEVADTEGAPVASVGSL | |
| | MMRPADPAKLGGARDGHHDALFRMEWVSRAVRVADGTPVGPWALVGDDG | |
| | LGLGTVLDAAQMRRYADLTALTGEFEELATTDSADTFDDSRPELVLFCH | |
| | LPGEGAASGDPAAARVELFKALSLVRSWLADERFAGTRLAVVTRGAVAA | |
| | DDAEQPDLASAPVWGLLRTAQTENPDRFVLVDVDEDERSLRALPAALAC | |
| | GEPQFAVRGGEVLVPRLVRAGSAADGAPTPPRERAVGRPDATMPSVWDP | |
| | EGTVLITGGTGTLGGRFARHLVTEYGVRRLLLVSRRGPGAEGAAELVER | |
| | LAGLGATATAVACDVSDPEALSGLLEAIPAEHPLTAVVHTAGVLDDGLV | |
| | SALTPERMDAVLRPKADAAWHLHRLTQGRDLRAFVLFSSVMGALGGAGQ | |
| | GNYAAANVYLDALAALRRAQGLPATSLAWGFWDERSELTGDLDQADLAR | |
| | MARAGLVPLRSDEGLALFDTALALGEPTLVPARLDTTRLAKDGNGPLPA | |
| | VLGALVRPRAARRTAAAGSAGGATGGQRFAGMSAADAERELMEAVRAHT | |
| | ATVLGHATPEAVRPDSRFKDIGFDSLSSVELRNRLSAAFGLRLPATAVF | |
| | DHPTPATMARHLRGEL (SEQ ID NO: 23) | |
| AztAD10 | VAIVGMGCRLPGGVTSPEELWELVASGSHGISGLPTDRGWDVDGLYDPD | SEQ ID |
| (module 3) | PDRRGKSYVREAGFLYDAGEFDADFFGIAPREALAMDPQQRLLLETTWE | NO: 20 |
| | AFERAGIRPESVHGSRTGVFVGAMPQEYGPHLHDATEGLDGLLLTGNTT | |
| | SVLSGRLAYFLGLEGPALTIDTACSSSLVALHQAAHALRQGECTLAVAG | |
| | GVAVMATPGVLTEFSRQRALAPDGRIKSFAASADGTGWSEGVGILLLER | |
| | LSDARKNGHQVLAVVRGSAVNQDGASNGLTAPNGPSQERVIRQALANAR | |
| | LTPDEVDVVEAHGTGTKLGDPIEAQALIATYGQNRPEDRPLWLGSLKSN | |
| | IGHSMAAAGVSGVIKMVMALRHGVLPRTLNVDEPTPHVDWPAGAVELLT | |
| | EERSWPDPGRPRRAAVSSFGISGTNAHLIVEQAPEDTGVTREGVPEPEV | |
| | VTDGTVPWLVSAKSEAALAEQARRLLDHLDRHPGVTPAEVGHTLAASRS | |
| | VFDHRAVLIGRELADHRGALTALAAGEPSARVVTGTVAAQPGRTVFVFP | |
| | GQGSQWAGMGVELMRTSPVFAEHLTACAAALEPHTGWSLIDVLDQTQGA | |
| | PDLDRVDVIQPALWAMMISLARLWEHLGITPDAVVGHSQGEIAAAHIAG | |
| | VLSLEDSARIVALRSRAITHIAGDGGMVSLPLAVADAEELIARWEERVV | |
| | VAAVNGPSATVVAGDADALAEIVTHCEGEDIRARKIPVDYASHSPHVEA | |
| | LHDELLELLAPVRPREAEVAFYSTVGDHAKGAMSDTTAMGAAYWYENLR | |
| | TTVAFEAAARALLDDGHTLFVEVSPHPVLTHPLQETVEDHTGAGEVAVT | |
| | GTLRRDDDTWQRVLTALATAHTHTTAPVDWSGFFPATRPTHLDLPTYPF | |
| | QHQHYWIQQTTTATDPHTLGLHAADHGLLGAAVALADGDGHVFTGHLSL | |
| | RSHPWLADHAVHDTSLLPGTAFVELALHAGQATDTPHLEDLTLEAPLTL | |
| | PATGGLHLQVHVAASDGDGHRALTIHSRPDDATPDLPWTRHANGTLAPQ | |
| | PSGAPDPAEWAELAAWPPAGATPVPAGTLYDHLADRGYRYGTTFQGLTA | |
| | VWRQGGALYAEVTLSDDTDNATAADHYGIHPALFDAALHPIVGAGPEQD | |
| | SDQVLLPFAWSNVQLHAVGARALRVQISPADAGTLRVRLADPTGQPVAE | |
| | AASLALRPITTEQLAKAVAAGGDDHLFRLAWTPASVTEELKTGRVAFLG | |
| | AAVPEALVTSLPGDVAVERHEKLAPLLADDTALLPDLVIATGLLERSHS | |
| | GEDVPGPAREAVQYALDLVQEWLAEERLAGSRLVFVTRRAVAVHADTES | |
| | PSPADAAVWGLIRTAEAENPGRFTLIDLADEKAIASESFRAALGSGEPQ | |
| | VAVRDGDQRLYVPRLVREVQPDDTAVPEPAAGGTVLITGGTGTLGTLFA | |
| | RHYATARQAGHLLLTSRRGPDAPGARELAAELTELGVKVTVVACDTADR | |
| | GALAALLAAVPDEHPLTAVVHTAGVLDDGTITSLTAERVERVFRPKVDA | |
| | AWHLHELTRESDLTEFVLFSSAAGVLGTPGQGNYAAANVELDALAERRR | |
| | ADGLPATSLAWGLWSDSSGMTGHLDDVDLTRMARLGIKPITAEEGVALF | |
| | EAARATGAACLVPAKIAPALLRPHLETGTLPAVLQGLVRAPVRKATVAT | |
| | ATDGATLRDRLAHLSPEEAEDTLATLVRTHVALVLGHDTSDTISLDKAF | |
| | KDLGFDSLTAVELRNRLASSTGLSLAATLVFSYPTPRELGRHLHDL | |
| | (SEQ ID NO: 24) | |
| AztAE10 | IAVVAMACRYPGGVSSPEELWNVVRDGLDVVGEFPQDRGWRDIFDPDPD | SEQ ID |
| (module 4) | TLGSSYTRHGGFLTDAAAFDAGFFGISPREALAVDPQQRLLLETSWEVE | NO: 21 |
| | ERAGIVPADVRGADVGVFSGVSSAEYGTRFVEASGHDLEGYLLQGSVLS | |
| | IASGRVAYEFGLTGPAVSVDTACSSSLVAVHLAMQSLRSGESSLALAGG | |
| | AAVMATPALFVEFSRQRGLSADGRCRSFADAADGTGWAEGVGVLLLERL | |
| | SDARRNGHPVLAVLRGSAVNQDGASNGLTAPNGRAQEKVIRKALANAGV | |
| | SAAEVDLLEAHGTGTTLGDPIEAGALLATYGQGRAEGRPLRLGSLKSNI | |
| | GHSMAAAGVGGVIKAVMAMRHRYLPKTLHVDQPSRHVDWSSGALELLLE | |
| | GREWTRAGGPRRAAVSSFGVSGTNAHVILEEAPRQEQADEGDPDSGVVG | |
| | GLVPWALSGKSAAAVQQQARKLREFAVADPGLDVADVGWSLTSSRTRFG | |
| | HRAVVLGHDRDELLSGLTALAAGEESAAVVRGTARELGGTVFMFPGQGS | |
| | QWVGMGRQLYDTFPVFAQSLDACAAALAEWVDWSLLDVVRGVEGAPTLD | |
| | RVDVVQPALFSVMVSMAALWRSWGVEPAAVVGHSQGEIAAAHVCGALSL | |
| | RDAAKVVALRSKALVDLIGHGGMASVAESADVVAERLAPWSDRVSIAVV | |
| | NGPRSVVVSGEPDALDEFVEKMKAEGAQARRIKVDYASHSHHVARVRDQ | |
| | VLGPLSDMSPKTSTLPFYSTLYGEVIDTAQLNGDYWYTNLREKVVFETS | |
| | VRRLADDGFRVFIEMSPHPVLTVPVQEIVEDVDDAVVLSSSRRDRGEVE | |
| | AVLGSLAGLHVQGGSVDWDVLFGTRRRVDLPTYAFQRQRYWLNSVHTGV | |
| | TAEVSALEPPAADDDPVALPDLIADLSDDEATALVLDHVLTKVAVVLGH | |
| | PSSEAVDPDQEFKDIGFDSLLSVQLSKGLATTTGLKLRPNLVLRHPTPR | |
| | RVAGYLKTS (SEQ ID NO: 25) | |

Polyketide synthases (PKS) are a family of multi-domain enzymes or enzyme complexes that produce polyketides, a large class of secondary metabolites, in bacteria, fungi, plants, and a few animal lineages. PKS genes for an individual polyketide are usually organized in single operon or in gene cluster. PKSs can be classified into three groups: Type I, Type II and Type II. Type I polyketide synthases are large, highly modular proteins, Type II polyketide synthases are aggregates of monofunctional proteins, and Type III polyketide synthases do not use Acyl carrier protein (ACP) domains. All Types of PKSs, and modules thereof, capable of contributing to the production of the compound of Formula (I) are envisaged as within the scope of the instant disclosure.

Type I polyketide-synthase modules comprise several domains with defined functions, separated by short spacer regions. An exemplary, but non-limiting Type I PKS protein comprises, from N to C terminus, a starting or loading module comprising an Acyltransferase (AT) and Acyl carrier protein (ACP) domain, an elongation or extending module comprising Keto-synthase (KS), AT, Dehydratase (DH), Enoylreductase (ER) and Ketoreductase (KR) domains, and a termination or releasing domain or module comprising a Thioesterase. As the polyketide is synthesized, the nascent polyketide chain is passed from one thiol group to the next by trans-acylation reactions, and is released at the end by hydrolysis or cyclization. At the start, the starter group, for example acetyl-CoA or an analogue thereof, is loaded onto the ACP domain of the starter module in a reaction catalyzed by the starter module's AT domain. In the polyketide elongation stages, the nascent polyketide chain is passed from the ACP domain of the previous module to the KS domain of the current module, in a reaction catalyzed by the KS domain. The elongation group, usually malonyl-CoA or methylmalonyl-CoA, is loaded onto the current ACP domain in a reaction catalyzed by the current AT domain. The ACP-bound elongation group reacts in a Claisen condensation with the KS-bound polyketide chain under CO2 evolution, leaving a free KS domain and an ACP-bound elongated polyketide chain. The reaction takes place at the KSn-bound end of the chain, so that the chain moves out one position and the elongation group becomes the new bound group. In some cases, the fragment of the polyketide chain can be altered stepwise by additional domains. This cycle is repeated for each elongation module, until finally the TE domain hydrolyzes the completed polyketide chain from the ACP-domain of the previous module.

In some embodiments, the biosynthetic gene cluster comprises a sequence encoding one or more PKS modules. In some embodiments, the sequence encoding the PKS module is selected from the group consisting of SEQ ID NOS: 18-21, or a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, the sequence encoding the PKS module comprises, or consisting essentially of, a sequence selected from the group consisting of SEQ ID NOS: 18-21.

In some embodiments, the biosynthetic gene cluster comprises sequences encoding four PKS modules. In some embodiments, the sequences encoding the four PKS modules comprise sequences of SEQ ID NOS: 18-21, or sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, the sequences encoding four PKS modules comprise sequences of SEQ ID NOS: 18-21.

In some embodiments, biosynthetic gene cluster comprises one or PKS modules comprising a sequence selected from the group consisting of SEQ ID NOS: 22-25, or a sequence having at least 90%, at least 95%, at least 97%, or at least 99% identity thereto. In some embodiments, biosynthetic gene cluster comprises one or PKS modules comprising a sequence selected from the group consisting of SEQ ID NOS: 22-25. In some embodiments, biosynthetic gene cluster comprises four PKS modules comprising sequences of SEQ ID NOS: 22-25, or sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, biosynthetic gene cluster comprises four PKS modules comprising sequences of SEQ ID NOS: 22-25.

In some embodiments, the four PKS modules are organized in 1, 2, 3 or 4 open reading frames. In some embodiments, the open reading frames comprise sequences selected from the group consisting of SEQ ID NOS: 26 and 27, or sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity thereto. In some embodiments, the four KPS modules are organized in two open reading frames. In some embodiments, the four KPS modules are encoded by sequences comprising a first KPS open reading frame of SEQ ID NO:26, and a second KPS open reading frame of SEQ ID NO: 27, or sequences having at least 90%, at least 95%, at least 97% or at least 99% identity thereto. In some embodiments, the four KPS modules are encoded by sequences comprising SEQ ID NOS: 26-27.

The biosynthetic gene cluster of the disclosure can comprise additional genes involved in the production of the compound of Formula (I), in addition to genes encoding NRPS and KPS proteins or modules. For example, the biosynthetic gene cluster can include genes that regulate the expression of other genes in the cluster, such as NRPS and KPS encoding genes, genes involved in the synthesis of precursor compounds that are involved in the synthesis of the compound of Formula (I), and genes involved the transport of same.

In some embodiments, the biosynthetic gene cluster further comprises a sequence encoding a Streptomyces Antibiotic Regulatory Protein (SARP), sometimes referred to herein as the SARP-encoding gene. SARP-family regulators are transcription factors that are activators of biosynthesis pathways in streptomycetes and actinomycetes bacterial species, for example antibiotic biosynthesis. SARP genes are known to occur in various types of antibiotic gene clusters encoding, inter alia genes encoding for the synthesis of polyketides, non-ribosomally synthesized peptides, and beta-lactam antibiotics, where they can regulate expression of genes in the gene cluster. SARP proteins are characterized by winged helix-turn-helix DNA binding motif at the N-terminus that binds to a conserved recognition motif within the major groove of target DNA. An exemplary SARP recognition motif comprises direct heptameric repeat sequences followed by 4 bp spacers, which are often localized between the −10 and the −35 promoter element of the respective target DNA.

In some embodiments, the biosynthetic gene cluster comprises a SARP-encoding gene comprising a protein coding sequence of SEQ ID NO: 28, or a sequence having at least 90%, at least 95%, at least 97% or at least 99% identity thereto. In some embodiments, the biosynthetic gene cluster comprises a SARP-encoding gene comprising a protein coding sequence comprising, or consisting essentially of SEQ ID NO: 28.

In some embodiments, the biosynthetic gene cluster comprises a sequence encoding a SARP protein comprising a sequence of:

| (SEQ ID NO: 29) | |
|---|---|
| 1 | MLGPFEMRTD DGVWVDVPGA RLRGLLIALA LRPGQVVPKA SLVDWIWGEQ SPADATNALQ |
| 61 | RLVSRLRKAL PYGAVEGHTD GYRLIVEPDA VDAVRFERLV VASQARTENA SRRAQLLREA |
| 121 | LELWRGAAMQ DSGPQDSAAF DAAVVRLDGL RLTALEEWED AENTLGRGAE LVTELTDLVA |
| 181 | AHPLRERLVA ALMRALVAAG SDSQALLVYQ RAKEALADAL GVDPSPELSA LHVALLRGEL |
| 241 | GTRERNRKTN LRAELTSFIG KGADVAAVRE LIAEHRLTTV IGPGGAGKTR LAAETARAML |
| 301 | GDLPDGAWLV ELAAIGADGD VADVAQATLA GLGLRDALLG GAPNVELTDR LIAAIREREA |
| 361 | LLILDNCEHV VESAAVFAHR VLGECRRLRI LATSREPLGI TGEALWQADP LALPEPGASP |
| 421 | DEIESAPAVR LLRDRAGAVR RDLASDARTL ATMARVCRAL DGMPLAIELA AARLRTMSID |
| 481 | QLAHRLDDRF RLLTSGSRTA LPRHKTLRAL VDWSWELLTD AERLVLRRLS VFSGGASLDA |
| 541 | AERVCAGAAV EQEQVLELLT SLTEKSLLRA EGDSAPRYRM IGTIKEYAGQ RLAEAGEAEL |
| 601 | ARHAHLACFT ELAETAEPHL RRAEQLKWLA TLEAEHDNIG AAMRGALAAG EAQAAMRLAA |
| 661 | GAGWYWWLGG HRSEGLELIT AASRMPGEVA DEVRAVMYAL VVHFLSSGPG DEHQVAEWID |
| 721 | KAYRFSRHSR RSHPLMGFIA PLKRMLQGPD AFLPAFEPLL DDEDPWARAL ARLHLGKMRI |
| 781 | MLGQGGRDVD AHLERALAEF RAIGERFGIS FALTELADRI AARGEFTAAC EHYEQAIAVV |
| 841 | TEVGAIEDII QMRSRQAQLY WLLGDEDASA AAIAEAQRYA ERVAWPGALA VLALSKAELA |
| 901 | RWGGRPEEAR RQLGAATALL GDDAEQANIR AVTHDLLGYL ADDLGEARAY RAAACAAASE |
| 961 | AGHAPLIARA LVGVADLALR RDQPEQAARL LAASTSVRGL ADRSHTDVAR IEQTARRRLG |
| 1021 | DAGEVEAARE GTRTSWSQLV EVTLAS, |

or a sequence having at least 90%, at least 95%, at least 97% or at least 99% identity thereto. In some embodiments, the biosynthetic gene cluster comprises a sequence encoding a SARP protein comprising, or consisting essentially of SEQ ID NO: 29.

In some embodiments, the biosynthetic gene cluster comprises an mbtH gene. mbtH proteins are a family of small proteins encoded by genes found in many, but not all, non-ribosomal peptide synthetase-encoding gene clusters. Approximately 70 amino acids in length, mbtH proteins are named after mbtH contained in the gene cluster for the siderophore mycobactin in Mycobacterium tuberculosis, which codes for a 71-amino acid protein. Without wishing to be bound by theory, it is thought that mbtH genes are involved in the biosynthesis pathways of the gene clusters in which they reside.

In some embodiments, the biosynthetic gene cluster comprises an mbtH gene. In some embodiments the biosynthetic gene cluster comprises four NRPS open reading frames, and the mbtH gene us located upstream of the four NRPS open reading frames. In some embodiments, the mbtH gene comprises a coding sequence comprising a sequence of SEQ ID NO: 30, or a sequence having at least 90%, at least 95%, at least 97% or at least 99% identity thereto. In some embodiments, the mbtH coding sequence comprises, or consists essentially of, SEQ ID NO: 30. In some embodiments, the mbtH protein comprises a sequence of SEQ ID NO: 31, or a sequence having at least 90%, at least 95%, at least 97% or at least 99% identity thereto. In some embodiments, the mbtH protein comprises a sequence of SEQ ID NO: 31.

In some embodiments, the biosynthetic gene cluster comprises an ornithine monooxygenase gene, and two PKS open reading frames. In some embodiments, the ornithine monooxygenase gene is downstream of the second PKS open reading frame. In some embodiments, the ornithine monooxygenase gene comprises an ornithineN-monooxygenase. In some embodiments, the ornithine N-monooxygenase gene comprises a coding sequence of SEQ ID NO: 32, or a sequence having at least 90%, at least 95%, at least 97% or at least 99% identity thereto. In some embodiments, the ornithine N-monooxygenase gene comprises a coding sequence that comprises, or consists essentially of SEQ ID NO: 32.

In some embodiments, the biosynthetic gene cluster is a wild type biosynthetic gene cluster isolated or derived from Streptomyces griseochromogenes. In some embodiments, the biosynthetic gene cluster comprises 6 NRPS modules encoded by sequences comprising SEQ ID NOS: 2-7, and 4 KPS modules encoded by sequences comprising SEQ ID NOS: 18-21. In some embodiments, the 6 NRPS modules are arranged in 4 open reading frames comprising sequences of SEQ ID NOS: 14-17, and the 4 KPS are arranged in 2 open reading frames comprising sequences of SEQ ID NOS: 26-27. In some embodiments, the biosynthetic gene cluster further comprises a SARP-encoding gene comprising a sequence of SEQ ID NO: 28, which is located upstream of the 2 KPS open reading frames, for example as shown in FIG. 2A.

In some embodiments, the biosynthetic gene cluster comprises a sequence of SEQ ID NO: 1, or a sequence having at least 90%, at least 95%, at least 97% or at least 99% identity thereto. In some embodiments, the biosynthetic gene cluster comprises a sequence of SEQ ID NO: 1. In some embodiments, the biosynthetic gene cluster consists essentially of a sequence of SEQ ID NO: 1.

In some embodiments, one or more genes of the biosynthetic gene cluster are expressed by a host cell comprising the biosynthetic gene cluster, resulting the production of the compound of Formula (I). In some embodiments, the host cells is engineered to express one or more genes in the biosynthetic cluster, which results in the production of the compound of Formula (I).

In some embodiments, overexpression of one or more genes in the biosynthetic cluster by the host cell increases the production of the compound of Formula (I) compared to an otherwise equivalent host cell comprising a biosynthetic gene cluster that does not overexpress one or more genes in the biosynthetic cluster.

In some embodiments, the modified host cell increases the production of the compound of Formula (I) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100% as measured by LCMS.

In some embodiments, the host cell overexpresses the SARP-encoding gene. SARP can be overexpressed in cis or in trans. For example, for cis over expression, the promoter of the SARP protein in the biosynthetic gene cluster can be modified to increase SARP expression. Alternatively, or in addition, SARP can be expressed in trans to the biosynthetic gene cluster by the host cell, for example by using a strong promoter to drive SARP expression.

Without wishing to be bound by theory, it is thought that the SARP protein regulates the expression of additional genes in the biosynthetic gene cluster by acting as a transcriptional activator. Increasing the expression of SARP increases the expression of SARP target genes in the biosynthetic gene cluster, thereby increasing the production of the compound of Formula (I) by the host cell.

In some embodiments, increasing the expression of SARP increases the production of the compound of Formula (I) by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100% as measured by LCMS.

The disclosure provides polynucleotides comprising a sequence encoding the SARP protein and a sequence of a promoter, for the overexpression of SARP protein in a host cell. The SARP protein can be expressed in trans in a host cell, from a polynucleotide that does not form a part of the biosynthetic gene cluster. For example, the host cell can comprise a first vector comprising the biosynthetic gene cluster, and a second vector comprising the sequences of the SARP protein and a promoter.

As used herein, “promoter” refers to a DNA sequence capable of controlling the expression of a coding sequence or functional RNA.

In some embodiments of the polynucleotides comprising a sequence encoding the SARP protein and a sequence of promoter, the two are operably linked. In some embodiments, the sequence encoding the SARP protein comprises a sequence of SEQ ID NO: 28, and the promoter comprises a sequence of SEQ ID NOS: 33-36 as set forth in Table 3.

Representative promoters that can be used to overexpress genes such as SARP, either in cis by insertion into the BGC, or in trans, are presented in Table 3 below.

TABLE 3
Promoter Sequences

| Name | DNA Sequence |
|---|---|
| ermE* | AGGTGCACGCGGTCGATCTTGACGGCTGGCGAGAGGTGCGGGGAGGATCTGACCGACGCG |
| | (SEQ ID NO: 33) |
| KasO* | AACTCCCCCAGTCCTGCACGCTGTCGTATTCTCCTGGCCACGACTTTACAACACCGCACAGC |
| | ATGTTGTCAAAGCAGAGACCGTTCGAATGTGAACA (SEQ ID NO: 34) |
| gapdh | GCGTATCCCCTTTCAGATACTCGCACTAAGAATTGCAGGAACGCCCCGATCATAGCGGTAGC |
| | CGCCCAGATGCTGCAAGCCTTATCTGGCAGCCGTATAAAAAAAGCAACCGAACAGGCCATTC |
| | ACAGAAGTTTCACACCGCTCGCCGAGGGGCTCCGCACCCGGTGCGCGCCGTACAGCAGCGCG |
| | CTTCTCCCAGCATCGGCCAGTTCGCCCGATCCGTCCGTGTCGCACAGCCGACGGCTGCGGTA |
| | AGGTGCCCGTAGACGCACGTCCGACCGAAGGAGCAGC (SEQ ID NO: 35) |
| rpslp | GCCCCGGGCCGGAGGCGGCCGCGACCACGACGCCCGCGGGACGTGACGAGCGGCACGACTCG |
| | ACGACTCCGGGCTCCTTTGACGCTGTCCGTCGCGCCGGGTAGCGTAGGACACCGTGCCCGCG |
| | CCGTCGGGCCCTCGCGCGTGCACTCGGTCGACCGCTCCCTGCCGGAGTGGGTGCGGGTGCAC |
| | GGGGTGGCTCCCCACCTCCTCTCGGATCGGTCCTCGCGGACTGCCGCCGTGCGGAGGACCGG |
| | GGCGACACGCCCGGGCGCGGGGGTCGGTGCGGGACTCCAGACCTCCGGGGTAGTCGTGCGAC |
| | GGGCGACGATCCGGGCCGAGCCGGCCGTCCTGGGTGACGGGTGCCGGTCAGACCAGAGAACA |
| | CCGACAGACGGAGACGTA (SEQ ID NO: 36) |

In some embodiments, trans overexpression of the SARP-encoding gene comprises expressing the SARP-encoding gene under the control of an ermE promoter, a kasO promoter, a gapdh promoter, a rpslp promoter, or a functional variant or derivative thereof. In some embodiments, trans overexpression of the SARP-encoding gene comprises expressing the SARP-encoding gene under the control of a constitutive ermE promoter, or a functional variant or derivative thereof. In some embodiments, the ermE promoter is an ermE* promoter comprising a sequence of SEQ ID NO: 33.

### Modified Biosynthetic Gene Clusters

The disclosure provides polynucleotides comprising biosynthetic gene clusters that have been modified relative to their wild type, or native equivalents, to increase production of the compound of Formula (I) when the genes of the biosynthetic gene cluster are expressed by a host cell.

In some embodiments, the modified polynucleotide increases the production of the compound of Formula (I) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100% as measured by LCMS.

Modifications of the biosynthetic gene cluster can be modified relative to a biosynthetic gene cluster comprising a sequence of SEQ ID NO: 1, or a sequence having at least 95%, at least 97% or at least 99% identity thereto. In some embodiments, the modification comprises one or more modifications relative to a sequence of SEQ ID NO: 1.

In some embodiments, the biosynthetic gene cluster is modified to overexpress the SARP protein in a host cell, thereby increasing production of the compound of Formula (I) by the host cell. In some embodiments, the at least one modification of the biosynthetic gene cluster comprises a modification that results in overexpression of the SARP-encoding gene in comparison to the expression of the SARP-encoding gene by the biosynthetic gene cluster of SEQ ID NO: 1.

All modifications are envisaged as within the scope of the instant disclosure. Exemplary, but non-limiting, modifications include substitutions, deletions, inversions, or insertions of heterologous sequences. In some embodiments, the one or more modifications of the biosynthetic gene cluster comprises a substitution, deletion, inversion, or insertion of one or more nucleotides relative to SEQ ID NO: 1.

In some embodiments, the one or more modifications comprise modifications of a promoter of a gene in the biosynthetic gene cluster. For example, a heterologous promoter sequence can be inserted near the coding sequence of one or more genes of the BGC. Alternatively, or in addition, one or more promoters of a gene in the BGC can be replaced with a heterologous promoter. Heterologous promoters include, inter alia, strong promoters, constitutive promoters and regulatable promoters. Exemplary strong promoters include ermE* (SEQ ID NO: 33), kasO (SEQ ID NO: 34), gapdh (SEQ ID NO: 35) and rpslp (SEQ ID NO: 36) as shown in Table 3. In some embodiments, replacement of one or more promoters comprises replacement of the SARP promoter, for example with a promoter shown in Table 3.

In some embodiments, the one or more modifications comprise insertion of at least one heterologous promoter in the biosynthetic gene cluster of SEQ ID NO: 1. In some embodiments, the at least one heterologous promoter is a strong promoter. In some embodiments, the at least one heterologous promoter is selected from the group consisting of ermE, kasO, gapdh, and rpslp, or functional variants or derivatives thereof. In some embodiments, the at least one heterologous promoter comprises a sequence of SEQ ID NOS: 33-36, or a functional variant or derivative thereof. For example, engineered versions of the ermE and kasO promoters used herein are sometimes referred to herein as ermE* and kasO*. In some embodiments, the at least one heterologous promoter comprises a sequence of SEQ ID NOS: 33-36, or a sequence having at least 90%, at least 95%, at least 97% or at least 99% identity thereto.

In some embodiments, the one or more modifications comprise insertion of at least one promoter in the biosynthetic gene cluster. In some embodiments, the at least one promoter is inserted upstream of the mbtH gene. For example, an ermE* promoter of SEQ ID NO: 33 or a kasO* promoter of SEQ ID NO: 34 is inserted upstream of the mbtH gene in SEQID NO: 1. Alternatively, or in addition, the at least one promoter is inserted between the SARP-encoding gene and the first PKS open reading frame in the biosynthetic gene cluster of SEQ ID NO: 1. For example, an rpslp promoter of SEQ ID NO: 36 or a kasO* promoter of SEQ ID NO: 34 is inserted between the SARP-encoding gene and the first PKS open reading frame in SEQ ID NO: 1. Alternatively, or in addition, the at least one promoter is inserted downstream of the second PKS open reading frame and upstream of the ornithine monooxygenase gene of SEQ ID NO: 1. For example, an gapdh promoter of SEQ ID NO: 35 is inserted downstream of the second PKS open reading frame and upstream of the ornithine monooxygenase gene of SEQ ID NO: 1.

In some embodiments the biosynthetic gene cluster comprising one or more modifications relative to SEQ ID NO: 1. In some embodiments the modified biosynthetic gene cluster comprises SEQ ID NO: 49 or 50.

### Methods of Modifying Biosynthetic Gene Clusters

The disclosure provides methods of modifying the biosynthetic gene clusters described herein. In some embodiments, methods of modifying the biosynthetic gene clusters described herein comprise a nucleic acid guided endonuclease.

The disclosure provides methods of modifying biosynthetic gene clusters comprising (a) providing a first E. coli host cell comprising a first vector comprising a sequence of an unmodified biosynthetic gene cluster comprising a target sequence; (b) introducing the first vector into a Streptomyces host cell by conjugation; (c) providing a second E. coli host cell comprising a second vector comprising: (i) a sequence of at least one gNA specific to the target sequence operably linked to a promoter, (ii) a sequence encoding a Cas9 endonuclease; and (iii) a sequence encoding a donor template; and (d) introducing the second vector into a Streptomyces host cell by conjugation; whereby introducing the second vector into the Streptomyces host cell produces a double strand break in the target sequence and introduction of a donor template sequence, thereby generating a Streptomyces host cell comprising a modified biosynthetic gene cluster. In some embodiments, the unmodified gene cluster comprises SEQ ID NO: 1, and the one or more modifications are modifications of SEQ ID NO: 1.

In some embodiments, the nucleic acid guided endonuclease is a CRISPR/Cas endonuclease. In some embodiments, the CRISPR/Cas endonuclease is Cas9. Other endonucleases known in the art may be used with the constructs described herein.

As used herein, “CRISPR/Cas endonuclease” refers to an enzymatic system that includes a guide nucleic acid (gNA) contains a nucleotide sequence complementary or substantially complementary to a region of a target polynucleotide, and a protein with active Nuclease. The CRISPR/Cas systems include the CRISPR-Cas Type I system, the CRISPR-Cas Type II system, the CRISPR-Cas Type III system, and derivatives thereof. CRISPR/Cas systems include genetically modified nuclease systems and/or programmed nuclease systems derived from naturally occurring CRISPR-Cas systems. CRISPR-Cas systems can contain genetically modified Cas proteins and/or mutated Cas proteins. CRISPR/Cas systems may contain genetically modified and/or programmed gNA.

As used herein, the term “guide nucleic acid” or “gNA” refers to an NA that contains a sequence complementary or substantially complementary to a region of a target DNA sequence. A gNA may contain nucleotide sequences in a region other than the region complementary or substantially complementary to a region of a target DNA sequence, sometimes termed a leader RNA. A leader RNA can be an rRNA or a derivative thereof, for example, a rRNA: chimera RNAtracr. gNAs can be RNAs (gRNAs) or DNAs (gDNAs).

In the CRISPR/Cas systems described herein, the gNA forms a complex with the CRISPR/Cas enzyme and the targeting portion of the gNA targets the CRISPR/Cas endonuclease to a specific target sequence in a target DNA polynucleotide. The CRISPR/Cas endonuclease then cuts the DNA, producing a double strand break. This double strand break can be repaired by non-homologous end joining, resulting in a deletion, or by homology directed repair (HDR) from a donor template. If the donor template includes sequences different from the target DNA polynucleotide, these sequence differences are incorporated into the target DNA polynucleotide.

In some embodiments, the donor template comprises, from 5′ to 3′, a sequence homologous to a sequence 5′ of the target sequence, a sequence of a promoter, and sequence homologous to a sequence 3′ of the target sequence. In some embodiments, the promoter is selected from the group consisting of ermk, kasO, gapdh, and rpslp, or functional variants or derivatives thereof.

In some embodiments, the biosynthetic gene cluster comprises a sequence of SEQ ID NO: 1. In some embodiments, the sequence of SEQ ID NO: 1 is modified using a CRISPR/Cas endonuclease and a donor template to insert at least one heterologous promoter into the biosynthetic gene cluster. The heterologous at least one promoter can be inserted upstream of the mbtH gene in SEQ ID NO: 1, between the sequence of the SARP-encoding gene and the first PKS open reading frame in SEQ ID NO: 1, or downstream of the second PKS open reading frame and upstream of the ornithine monooxygenase gene in SEQ ID NO: 1.

Exemplary targeting sequences used to insert a promoter upstream of the mbtH gene are shown in Table 4 below. In some embodiments, the targeting sequence of the at least one gNA used to modify the BGC comprises SEQ ID NOS: 40-44, or a sequence having at least 80%, at least 85%, at least 90%, or at least 95% thereto. In some embodiments, the targeting sequence of the at least one gNA used to modify the BGC comprises SEQ ID NOS: 40-44.

TABLE 4
gNA Targeting sequences for mbtH

| Name | Sequence | SEQ ID NO |
|---|---|---|
| AZT010-sg1 | ATGAGGACCCTATCTGGGGG | 40 |
| AZT010-sg2 | CGAGTGCGCTCACTTCGGGG | 41 |
| AZT010-sg3 | CGAGCGGAGAGACGCCGGAA | 42 |
| AZT010-sg4 | GTGGCGTCAGCATTTCAGGG | 43 |
| AZT010-sg5 | GTCACGGGATGACTCGAAAG | 44 |

In some embodiments, gRNAs comprise a targeting sequence, sometimes referred to as a protospacer, and a scaffold. An exemplary sequence encoding a gRNA scaffold comprises a sequence of

GTTTTAGAGCTAGAAATAGCAAGTTAAAATAAGGCTAGTCCGTTATCAACTTGAA AAAGTGGCACCGAGTCGGTGC (SEQ ID NO: 45). An exemplary sequence encoding a Cas9 single gRNA (sgRNA) comprises, from 5′ to 3′, a targeting sequence complementary to a target sequence, and a scaffold sequence, for example SEQ ID NO: 45.

In some embodiments, the CRISPR/Cas endonuclease is a Cas9 endonuclease. An exemplary Cas9 endonuclease for use in the methods described herein comprises as sequence of SEQ ID NO: 47, and is encoded by a sequence of SEQ ID NO: 48.

In some embodiments, inserting at least one heterologous promoter into the biosynthetic gene cluster further comprises a donor template comprising a sequence of the heterologous promoter.

### Vectors

The disclosure provides vectors comprising the polynucleotides of the disclosure. In some embodiments, the vectors comprise the sequence of the biosynthetic gene cluster of SEQ ID NO: 1, or a sequence having at least at least 90%, at least 95%, at least 97% or at least 99% identity thereto. In some embodiments, the vector comprise the sequence of a biosynthetic gene cluster comprising at least one modification relative to SEQ ID NO: 1, for example the insertion of a heterologous promoter.

Suitable vectors for the cloning and expression of the biosynthetic gene clusters described herein will be known to persons of ordinary skill in the art. For example, suitable vectors for expressing biosynthetic gene clusters in Streptomyces are described in US20200291430A1, the contents of which are incorporated by reference in their entirety herein.

Exemplary vectors include, inter alia, cloning sites, promoters to direct expression of gene products, and selectable markers for host cells such as Streptomyces and/or E. coli. In some embodiments, the expression vector further comprises an E. coli and/or Streptomyces origin of replication. In some embodiments, the expression vector further comprises one or more selectable markers for E. coli and/or Streptomyces. A number of antibiotic resistance markers are available for Streptomyces, and include thiostrepton (tsr), kanamycin-neomycin (kmr), apramycin (amr), geneticin, viomycin, hygromycin, bleomycin, chloramphenicol, and the like.

In some embodiments, the expression vector further comprises a gene that stabilizes large plasmids. In some embodiments, the expression vector is configured to accept an insert comprising more than 10 kb, more than 20 kb, more than 50 kb, and/or more than 100 kb.

Suitable vectors can be configured to express a product of the biosynthetic gene cluster nucleic acid when the expression vector is present in a host cell, such as a Streptomyces host cell.

In some embodiments the vector is an expression vector.

In some embodiments the vector is a shuttle vector. As used herein, the term “shuttle vector” refers to a vector constructed so that it can propagate in two different host species, e.g., E. coli and another organism such as Streptomyces.

In some embodiments, the vector is a plasmid or a bacterial artificial chromosome.

### Synthesis of the Compounds of the Disclosure

In some embodiments, the compound of Formula (I) is synthesized using a semi-synthetic approach. In some embodiments, the compound of Formula (I) is synthesized using a biosynthetic approach.

In some embodiments, the compound is cyclized with the use of a biosynthetic gene cluster (BCG) such as the biosynthetic gene cluster described supra, sometimes referred to herein as the AZT010 biosynthetic gene cluster.

As used herein, “AZT010 biosynthetic gene cluster” refers to a biosynthetic gene cluster isolated or derived from Streptomyces species, which is described further detail supra. In some embodiments, the AZT010 biosynthetic gene cluster is isolated or derived from Streptomyces griseochromogenes. In some embodiments, the AZT010 biosynthetic gene cluster is isolated or derived from Streptomyces griseochromogenes strain ATCC 14511 (www.atcc.org/Products/All/14511?geo_country=us). The freeze-dried ATCC is not a pharmaceutical composition. The freeze-dried ATCC does not comprise detectable levels of a compound of Formula (I).

In some embodiments, the wild-type AZT010 biosynthetic gene cluster is modified. In some embodiments, the modified AZT010 biosynthetic gene cluster produces a compound of Formula (I). For the avoidance of doubt, the modification(s) of the BGC is necessary to produce quantifiable levels of the compounds of the disclosure. Modifications of the biosynthetic gene cluster can be carried out by any methods known in the art. For example, the BGC can be modified using a CRISPR/Cas endonuclease.

In some embodiments, the present disclosure provides a method of making a compound of Formula (I) comprising:
a. genome mining to identify a biosynthetic gene cluster;
b. modifying the identified biosynthetic gene cluster;
c. identifying a target compound; and
d. isolating the target compound.

In some embodiments, the genome mining identifies a biosynthetic gene cluster. In some embodiments, the identified biosynthetic gene cluster is AZT010. In some embodiments, AZT010 is isolated or derived from Streptomyces griseochromogenes strain ATCC 14511.

In some embodiments, the genome is sequenced prior to modification.

In some embodiments, the biosynthetic gene cluster is modified by overexpression of at least one gene in the cluster. In some embodiments, the overexpressed gene is SARP (Streptomyces Antibiotic Regulatory Protein).

In some embodiments, the biosynthetic gene cluster is isolated. In some embodiments, the biosynthetic gene cluster is isolated prior to identifying the target compound. In some embodiments, the biosynthetic gene cluster is isolated prior to isolating the target compound.

In some embodiments, the biosynthetic gene cluster is expressed in a heterologous host. In some embodiments, the heterologous host is S. albus.

In some embodiments, the biosynthetic gene cluster is further modified. In some embodiments, the biosynthetic gene cluster is further modified by the insertion of one or more strong promoters, using methods provided herein. In some embodiments, the strong promoter is kasO, or a functional derivative thereof.

In some embodiments, the compound of Formula (I) is isolated from culture.

In some embodiments, the compound of Formula (I) is isolated and then purified.

In some embodiments, the present disclosure provides a method of making a compound of Formula (I) further comprising a step of: (e) purifying the isolated compound.

In some embodiments, the present disclosure provides a method of making a compound of Formula (I), or derivatizing the compound of Formula (I), by solid phase peptide synthesis wherein the amino acid α-N-terminal is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. Other side chain protecting groups are, for example, for side chain amino groups (e.g., lysine and arginine) are 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine are benzyl, o-bromobenzyloxy-carbonyl, 2,6-dichlorobenzyl, isopropyl, t-butyl (t-Bu), cyclohexyl, cyclopentyl and acetyl (Ac); for serine are t-butyl, benzyl and tetrahydropyranyl; for histidine are trityl, benzyl, Cbz, p-toluenesulfonyl and 2,4-dinitrophenyl; for tryptophan are formyl; for aspartic acid and glutamic acid are benzyl and t-butyl; and for cysteine are triphenylmethyl (trityl). In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides may be 4-hydroxymethylphenoxymethyl-copoly(styrene-1% divinylbenzene) or 4-(2′,4′-dimethoxyphenyl-Fmoc-aminomethyl)phenoxyacetamidoethyl. The α-C-terminal amino acid may be coupled to the resin by means of N,N′-dicyclohexylcarbodiimide (DCC), N,N′-diisopropylcarbodiimide (DIC), or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP), or bis(2-oxo-3-oxazolidinyl)phosphine chloride (BOPCI), mediated coupling for from about 1 hour to about 24 hours at a temperature of between 10° C. and 50° C. in a solvent (e.g., dichloromethane or DMF). When the solid support is 4-(2′,4′-dimethoxyphenyl-Fmoc-aminomethyl)phenoxy-acetamidoethyl resin, the Fmoc group is cleaved with a secondary amine (e.g., piperidine) prior to coupling with the α-C-terminal amino acid as described above. The coupling of successive protected amino acids may be carried out in an automatic polypeptide synthesizer. In some embodiments, the α-N-terminal in the amino acids of the growing peptide chain are protected with Fmoc. The removal of the Fmoc protecting group from the α-N-terminal side of the growing peptide may be accomplished by treatment with a secondary amine (e.g., piperidine). Each protected amino acid may then be introduced in about 3-fold molar excess, and the coupling may be carried out in DMF. Following completion of synthesis, the polypeptide is removed from the resin and deprotected, either in successively or in a single operation. Removal of the polypeptide and deprotection may be accomplished in a single operation by treating the resin-bound polypeptide with a cleavage reagent (e.g., thianisole, water, ethanedithiol, and trifluoroacetic acid). In cases wherein the α-C-terminal of the polypeptide is an alkylamide, the resin may be cleaved by aminolysis with an alkylamine. Alternatively, the peptide may be removed by transesterification (e.g. with methanol) followed by aminolysis or by direct transamidation. The protected peptide may be purified or taken directly to the next step without purification. The removal of the side chain protecting groups may be accomplished using the appropriate cleavage conditions. The fully deprotected peptide may be purified by a sequence of chromatographic steps employing one or more of the following types: ion exchange on a weakly basic resin (acetate form); hydrophobic adsorption chromatography on underivatized polystyrene-divinylbenzene (e.g., Amberlite XAD); silica gel adsorption chromatography; ion exchange chromatography on carboxymethylcellulose; partition chromatography (e.g., on Sephadex G-25, LH-20 or countercurrent distribution); high performance liquid chromatography (HPLC), such as reverse-phase HPLC on octyl- or octadecylsilyl-silica bonded phase column packing.

In some embodiments, compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art

One of ordinary skill in the art will note that, during the reaction sequences and synthetic scheme described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

Production of the Compounds of Formula (I) from Host Cells

The disclosure provides methods of making the compound of Formula (I) in a host cell comprising the biosynthetic gene cluster described herein.

In some embodiments, the host cell does not produce the compound of Formula (I) in the absence of the biosynthetic gene cluster described herein.

The disclosure provides methods of making the compound of Formula (I), comprising (a) introducing into a host cell the polynucleotides or vectors of the disclosure; (b) culturing the host cell under conditions sufficient for the synthesis of the compound of Formula (I) by the biosynthetic gene cluster; and (c) isolating and purifying the compound of Formula (I). In some embodiments, the host cell is a Streptomyces cell, such as a Streptomyces griseochromogenes or Streptomyces albus cell. In some embodiments, the host cell comprises a sequence encoding a SARP operably linked to a constitutive promoter.

Methods of introducing polynucleotides and vectors into suitable host cells will be known to persons of ordinary skill in the art, and include electroporation and by conjugation with an E. coli cell comprising the polynucleotide or vector.

Intergenic conjugation with E. coli allows for the introduction of vectors into Streptomyces species. Exemplary vectors for intergeneric conjugation between E. coli and Streptomyces comprise the 760-bp oriT fragment for conjugation, but require the transfer functions to be supplied in trans by the E. coli donor strain Some vectors include the attachment site (attP) and the integrase (int) function of the temperate phage φ31 to facilitate the site-specific integration of the vector at the affB site of the Streptomyces chromosome.

### Host Cells

The disclosure provides host cells. comprising the polynucleotides and vectors described herein.

In some embodiments, for example those embodiments where the BGC is an unmodified BGC of SEQ ID NO: 1, the host cell further comprises a polynucleotide comprising a sequence encoding a SARP operably linked to a constitutive promoter, such as ermE*. In some embodiments the sequence encoding the SARP comprises SEQ ID NO: 28.

The host cell, or host organism, is typically, but not necessarily, a genetically tractable (e.g., culturable under laboratory conditions and manipulable by molecular biological techniques) organism. The host organism may be a member of the domain Bacteria, the domain Eukarya, or the domain Archaea. In some embodiments, the host microorganism is from the domain Bacteria. In some embodiments, the host organism is a bacterium in the terrabacteria group. In particular embodiments, the host microorganism is from the taxa Actinobacteria, Streptomycetales, or Streptomycetaceae. In some embodiments, the host is from the genus Streptomyces. In some embodiments, the host is a Streptomyces expression strain, e.g., as defined herein (e.g. Streptomyces averminlis, Streptomyces venezuelae, Streptomyces albus, Streptomyces lividans, and Streptomyces coelicolor). In some embodiments, the host organism is a Streptomyces species. In some embodiments, the host is Streptomyces albus.

As used herein the term “Streptomyces expression strains” or “heterologous Streptomyces expression strains” refers to bacterial strains including, but not limited to, commonly used species such as Streptomyces avermitilis, Streptomyces venezuelae, Streptomyces albas, Streptomyces lividans, and Streptomyces coelicolor.

Methods of culturing host cells will be known to persons of ordinary skill in the art and are described in “Laboratory Maintenance of Streptomyces species,” Curr Protoc Microbiol. 2010 August; CHAPTER. Unit-10E.1, the contents of which are incorporated by reference in their entirety herein. For example, Streptomyces may be grown in suitable liquid media (e.g., Tryptic Soy-Broth (TSB), R2YE and YEME media) at about 28 ° C., in baffled Erlenmeyer or similar shaking flask systems. Long term storage of Streptomyces can be accomplished through glycerol stocks.

### Pharmaceutical Compositions

In some aspects, the present disclosure provides a pharmaceutical composition comprising the compound of Formula (I) as an active ingredient. In some embodiments, the present disclosure provides a pharmaceutical composition comprising the compound of Formula (I) and one or more pharmaceutically acceptable carriers, diluents or excipients. Pharmaceutically acceptable carriers, diluents or excipients include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

As used herein, the term “composition” is intended to encompass a product comprising the specified ingredients in the specified amounts, as well as any product which results, directly or indirectly, from combination of the specified ingredients in the specified amounts.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the compound of Formula (I)) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

The compounds, or pharmaceutically acceptable salts thereof, may be administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

The compound of Formula (I) can be formulated for oral administration in forms such as, for example, tablets, lozenges, hard or soft capsules, aqueous or oily suspensions, emulsions, dispersible powders, granules, syrups, elixirs, and tinctures. The compound of Formula (I) can also be formulated for intravenous (bolus or in-fusion), intraperitoneal, topical (for example as creams, ointments, gels, or aqueous or oily solutions or suspensions), inhalation (for example as a finely divided powder or a liquid aerosol), for administration by insufflation (for example as a finely divided powder), or parenteral administration (for example as a sterile aqueous or oily solution for intravenous, subcutaneous, intramuscular, intraperitoneal or intramuscular dosing) as a suppository for rectal dosing, or transdermal (e.g., patch).

In some embodiments, the present disclosure provides pharmaceutical compositions comprising a compound of Formula (I) combined with a pharmaceutically acceptable carrier. In some embodiments, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M phosphate buffer or saline (e.g., about 0.8%). Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated B-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT(r)), potassium chloride, powdered cellulose, sodium chloride, sorbitol, and talc.

In various embodiments, the pharmaceutical composition may be selected from the group consisting of a solid, powder, liquid and a gel. In certain embodiments, the pharmaceutical compositions of the present disclosure is a solid (e.g., a powder, tablet, a capsule, granulates, and/or aggregates). In certain of such embodiments, the solid pharmaceutical composition comprises one or more excipients known in the art, including, but not limited to, starches, sugars, diluents, granulating agents, lubricants, binders, and disintegrating agents.

In some embodiments, the pharmaceutical compositions of the present disclosure are prepared for oral administration. In certain of such embodiments, the pharmaceutical compositions are formulated by combining one or more agents and pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In some embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In some embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, the pharmaceutical compositions for oral administration are soft, sealed capsules made of gelatin and a plasticizer, such as glycerol or sorbitol. In certain soft capsules, one or more compounds disclosed herein, or a pharmaceutically acceptable solvate, hydrate, tautomer, N-oxide, or salt thereof, are be dissolved or suspended in suitable liquids, such as fatty oils, liquid paraffin, or liquid polyethylene glycols. In addition, stabilizers may be added.

Solid pharmaceutical compositions that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

Liquid pharmaceutical compositions can be prepared using compounds of the present disclosure, or a pharmaceutically acceptable solvate, hydrate, tautomer, N-oxide, or salt thereof, and any other solid excipients where the components are dissolved or suspended in a liquid carrier such as water, vegetable oil, alcohol, polyethylene glycol, propylene glycol, or glycerin.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Liquid pharmaceutical compositions can contain emulsifying agents to disperse uniformly throughout the composition and/or combination an active ingredient or other excipient that is not soluble in the liquid carrier. Emulsifying agents that may be useful in liquid compositions and/or combinations of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

In some embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

In certain embodiments, a pharmaceutical compositions of the present invention are formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

The amount of the compound of Formula (I) may be present in the composition in a therapeutically effective amount. For example, in some embodiments, the compound may be administered at about 0.001 mg/kg to about 100 mg/kg body weight (e.g., about 0.01 mg/kg to about 10 mg/kg or about 0.1 mg/kg to about 5 mg/kg).

A therapeutically effective amount of the compound of Formula (I) for use in therapy is an amount sufficient to treat or prevent cancer, slow its progression and/or reduce the symptoms associated with the condition.

A therapeutically effective amount of the compound of Formula (I) for use in therapy is an amount sufficient to treat cancer, slow its progression and/or reduce the symptoms associated with the condition.

A therapeutically effective amount of the compound of Formula (I) for use in therapy is an amount sufficient to treat or prevent fibrosis, slow its progression and/or reduce the symptoms associated with the condition.

A therapeutically effective amount of the compound of Formula (I) for use in therapy is an amount sufficient to treat fibrosis, slow its progression and/or reduce the symptoms associated with the condition.

The size of the dose for therapeutic or prophylactic purposes of Compound A will naturally vary according to the nature and severity of the conditions, the age and sex of the animal or patient and the route of administration, according to well-known principles of medicine.

Examples of useful dermatological compositions which can be used to deliver Compound A to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

### Methods of Use

A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human.

In some embodiments, the present disclosure provides a method of treating or preventing a disease or disorder disclosed herein in a subject in need thereof, comprising administering to the subject a therapeutically effective amount of the compound of Formula (I) or a pharmaceutical composition of the present disclosure.

In some embodiments, the present disclosure provides a method of treating cancer in a subject in need thereof, comprising administering to the subject a therapeutically effective amount of the compound of Formula (I) or a pharmaceutical composition of the present disclosure.

In some embodiments, the present disclosure provides a method of treating or preventing a disease or disorder disclosed herein in a subject in need thereof, comprising administering to the subject a compound of Formula (I) or a pharmaceutical composition of the present disclosure.

In some embodiments, the present disclosure provides a method of treating cancer in a subject in need thereof, comprising administering to the subject a compound of Formula (I) or a pharmaceutical composition of the present disclosure.

In some embodiments, the present disclosure provides a method of treating fibrosis in a subject in need thereof, comprising administering to the subject a therapeutically effective amount of the compound of Formula (I) or a pharmaceutical composition of the present disclosure.

In some embodiments, the present disclosure provides a method of treating fibrosis in a subject in need thereof, comprising administering to the subject a compound of Formula (I) or a pharmaceutical composition of the present disclosure.

In some embodiments, the present disclosure provides the compound of Formula (I) for use in treating cancer in a subject in need thereof.

In some embodiments, the present disclosure provides the compound of Formula (I) for use in treating fibrosis in a subject in need thereof.

In embodiments, the present disclosure provides use of the compound of Formula (I) in the manufacture of a medicament for treating a disease or disorder disclosed herein.

In some embodiments, the present disclosure provides use of the compound of Formula (I) in the manufacture of a medicament for treating cancer in a subject in need thereof.

In some embodiments, the present disclosure provides use of the compound of Formula (I) in the manufacture of a medicament for treating fibrosis in a subject in need thereof.

In some embodiments, the present disclosure provides use of the compound of Formula (I) for the treatment of a disease or disorder disclosed herein.

In some embodiments, the present disclosure provides use of the compound of Formula (I) for the treatment of cancer.

In some embodiments, the present disclosure provides use of the compound of Formula (I) for the treatment of fibrosis.

In some embodiments, the disease or disorder is a cancer.

In some embodiments, the cancer is a disease that involves abnormal cell growth with the potential to invade or spread to other parts of the body.

In some embodiments, the cancer is a malignant tumor or neoplasm.

In some embodiments, the cancer is breast cancer, pancreatic cancer, non-small cell lung cancer, ovarian cancer, esophageal cancer, melanoma, lymphoma, uterine cancer, peritoneal cancer, fallopian tube cancer, endometrial cancer, cervical cancer, thyroid cancer, gastric cancer, gastroesophageal junction cancer, urothelial cancer, bladder cancer, oropharynx cancer, hypopharynx cancer, larynx cancer, head and neck cancer, germ cell cancer/tumors, prostate cancer, colon cancer, rectal cancer, kidney cancer, cholangiocarcinoma (bile duct cancer), glioblastoma, leukemia, or non-Hodgkin lymphoma.

In some embodiments, the cancer is Acute Lymphoblastic Leukemia, Acute Myeloid Leukemia, Adrenocortical Carcinoma, AIDS-Related Cancers, Kaposi Sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Bone Cancer, Ewing Sarcoma Family of Tumors, Osteosarcoma and Malignant Fibrous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Primary, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Intraocular Melanoma, Islet Cell Tumors, Pancreatic Neuroendocrine Tumors, Kaposi Sarcoma, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Lymphoma, Cutaneous T-Cell (Mycosis Fungoides and Sézary Syndrome), Hodgkin Cancer, Non-Hodgkin Cancer, Macroglobulinemia, Waldenström, Male Breast Cancer, Malignant Fibrous Histiocytoma of Bone and Osteosarcoma, Melanoma, Intraocular (Eye) Cancer, Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia, Acute, Myeloma Multiple, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System Lymphoma, Rectal Cancer, Renal Cell (Kidney) Cancer, Retinoblastoma, Rhabdomyosarcoma, Salivary Gland Cancer, Sarcoma, Ewing Cancer, Kaposi Cancer, Osteosarcoma (Bone Cancer), Soft Tissue Cancer, Uterine Cancer, Sezary Syndrome, Skin Cancer, Childhood Melanoma, Merkel Cell Carcinoma, Nonmelanoma, Small Cell Lung Cancer, Small Intestine Cancer, Soft Tissue Sarcoma, Squamous Cell Carcinoma, Skin Cancer (Nonmelanoma), Childhood Squamous Neck Cancer with Occult Primary, Metastatic Cancer, Stomach (Gastric) Cancer, T-Cell Lymphoma, Cutaneous Cancer, Testicular Cancer, Throat Cancer, Thymoma and Thymic Carcinoma, Thyroid Cancer, Transitional Cell Cancer of the Renal Pelvis and Ureter, Unknown Primary, Carcinoma of Childhood, Unusual Cancers of Childhood, Urethral Cancer, Uterine Cancer, Endometrial Cancer, Uterine Sarcoma, Vaginal Cancer, Vulvar Cancer, Waldenström Macroglobulinemia, Wilms Tumor, and Women's Cancers.

In some embodiments, the disease or disorder is a fibrosis.

Fibrotic conditions are characterized, in whole or in part, by excess production of fibrotic material. These conditions can include systemic sclerosis, multifocal fibrosclerosis, nephrogenic systemic fibrosis, scleroderma (including morphea, generalized morphea, or linear scleroderma), sclerodermatous graft-vs-host-disease, kidney fibrosis (including glomerular sclerosis, renal tubulointerstitial fibrosis, progressive renal disease or diabetic nephropathy), cardiac fibrosis (e.g., myocardial fibrosis), pulmonary fibrosis (e.g. pulmonary fibrosis, glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, myelodysplastic syndrome, myeloproferative syndrome, collagenous colitis, acute fibrosis, organ specific fibrosis, and the like.

In some embodiments, the fibrosis is pulmonary fibrosis, liver fibrosis, heart fibrosis, mediastinal fibrosis, retroperitoneal cavity fibrosis, bone marrow fibrosis, or skin fibrosis.

In some embodiments, the fibrotic condition is pulmonary hypertension, chronic obstructive pulmonary disease (COPD), idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, or pulmonary hypertension,

In some embodiments, the fibrosis is cystic fibrosis.

In some embodiments, the subject is a mammal. In some embodiments the mammal is a human.

In some embodiments, the compound of Formula (I) is administered once, twice, three times, four times, or five times per day. In some embodiments, the compound of Formula (I) is administered once daily. In some embodiments, the compound of Formula (I) is administered twice daily. In some embodiments, the compound of Formula (I) is administered three times daily. In some embodiments, the compound of Formula (I) is administered four times daily. In some embodiments, the compound of Formula (I) is administered five times daily.

In some embodiments, the compound of Formula (I) is administered with a drug holiday. In some embodiments, the compound of Formula (I) is administered without a drug holiday.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present disclosure and practice the claimed methods. The following working examples therefore are not to be construed as limiting in any way the remainder of the disclosure.

### Example 1. Proposed Biosynthesis

Genome mining was performed using both public databases and internal sequence collection. A prioritized hit was found from the genome of Streptomyces griseochromogenes ATCC 14511.

Streptomyces griseochromogenes ATCC 14511 was obtained from the ATCC collection for verification of the biosynthetic gene cluster (BGC) by genome sequencing and identification of target compounds. The genome was sequenced using a combination of long-read (ONT) and short-read shotgun (Illumina) platforms. The target AZT010 BGC was identified in a 1.2 Mbp scaffold, confirming the presence and sequence of the gene cluster in the ATCC 14511 genome.

BGC analysis. The AZT010 gene cluster belongs to the type 1 modular hybrid NRPS-PKS family of BGCs. Six NRPS modules corresponding to the core peptide macrocycle are encoded in 4 open reading frames (aztN, aztO, aztP, and aztAG) (FIG. 1). The PKS contains 4 modules encoded in 2 open reading frames (aztAD and aztAE). Other genes for precursor biosynthesis, post-PKS modifications, regulation, and transport are distributed up and downstream of the NRPS and PKS core genes. aztN contains 7 typical domains belonging to 2 amino acid modules; module 1 contains C-A-T-E domains followed by C-A-T in module 2. aztO has 8 domains from modules 3 and 4; module 3 contains C-A-T-nMT-E domains followed by C-A-T from module 4. Module 5 is encoded in the aztP gene containing C-A-T-TE domains. aztAG downstream of the PKS contains module 6 of the NRPS core with a C (starter)-A-T domain. The presence of E domains (epimerization) in modules 1 and 3 predicted that these substrates are epimerized to D-amino acids in the final structure. From the substrate specificity for the A-domains (Stachelhaus codes and NRPSPredictor in Antismash 4/5), the peptide core of the compound was predicted to be ‘1: bOH-Leu (mod6)-2: piz (mod1)-3: nOHAla (mod2)-4: nMe-phe/trp (mod3)-5: ala/X(mod4)-6: gly/X (mod5)’, with weaker predictions for positions 4, 5, and 6. The PKS core is composed of four modules typical of the AZT BGCs. aztA) gene contains the first 3 modules—module 1 contains a KS-AT-ACP domain, followed by module 2 containing KS-AT-DH-KR-ACP domains, and module 3 with KS-AT-DH-KR-ACP. aztAE contains module 4 with KS-AT-ACP domains. Distinct from the other AZT BGCs, module 3 of the AZT010 PKS lacks an ER domain that corresponds to the saturated THP ring in the PKS tail.

Without being bound by theory, genes predicted to be involved in the biosynthesis or transformation of precursor amino acids were also identified in AZT010 BGC. azt (Z, AA, AB), homologous to ply(Q,R,S) in the polyoxypeptin biosynthesis, are proposed to be involved in b-OH leucine formation. azt AQ and CO (ktzI and kztT from kutzneride biosynthesis) are involved in the conversion of ornithine to piperazic acid. Azt (K, L., M) homologous to ply ((,D,E) are involved in the formation of hydroxamate containing residues. A set of precursor genes azt (('Q, CR, CS, (T, ('U, C′V) homologous to genes involved in the biosynthesis of cyclohexylalanine in salinosporamide and cinnabaramide BGCs are present in the AZT010 BGC. Based on the prediction of a bulky/ring residue in module 3, this position can be expected to take cyclohexylalanine as a substrate. Other biosynthetic genes identified in the gene cluster include a SARP regulator (aztT), an MbtH (aztI), and three sets of ABC transporters, (azt R/S, BA/BB, and DJ/DK).

### Example 2. Identification of Product/s of AZT010 BGC

Stable isotope labeling to detect AZTs. Two residues are conserved in most structures of the peptide core of azinothricin-like (AZTs) molecules: β-OH-leucine, and piperazic acid. The enzymatic routes and genes involved for the formation of these non-proteinogenic AAs are present in the AZT010 BGC. β-OH-leucine was proposed to be derived from L-leucine via hydroxylation by a cytochrome P450 enzyme (aztAA). The incorporation of D10-labeled β-OH-leucine in the compound core was detected as a shift of +8 Da in the MS spectra, consistent with the loss of 1H upon hydroxylation at the beta position, and a possible exchange of the acidic alpha proton during NRPS assembly. Piperazic acid residues are biosynthesized from L-ornithine by the action of two enzymes ktzT and ktzI as demonstrated in kutzneride biosynthesis. Homologs of ktzT and ktzI are found in AZT BGCs and this transformation was used as our secondary test for the presence and the number of Piz residues. This approach was validated in proof-of-concept experiments using strains producing known compounds verucopeptin and polyoxypeptin.

Small scale production and labeling experiments. A glycerol stock of AZT010 producer was scraped and inoculated into 10 mL sterile TSB (tryptic soy broth, 30 g/L) media in 50 mL aerated falcon tubes and grown for 2 to 3 days at 28° C., 220 rpm. 500 uL of this seed culture was inoculated into sterile 50 mL 1SF (glycerol 20 g/L, soybean flour 10 g/L, CaCO3 5 g/L) in 250 mL baffled flask. Cultures were grown for 7 days at 28° C., 220 rpm. On day 3 of production, filter-sterilized D10-leucine was added to the culture flasks to a final concentration of 3 mM. Cultures were grown in triplicates and harvested by extraction on day 7.

Extraction and LCMS analysis. Cultures were harvested by solvent extraction using 1:1 volumes of Chloroform: IPA (2:1) in 250 mL separatory funnel. The chloroform layer was dried, and the extracts were resuspended in 0.2 mL methanol. For LCMS analysis, 25 uL of the methanol extract was injected into a Phenomenex Kinetex C18 column (2.6 um, 100×4.6 mm). The following general condition was used for all LCMS profiling and analyses: flow rate, 1 mL/min, solvent gradient: 20-100% B in 1-20 mins, 100% B 20-25 min, ramp to 20% B 25-30 min. Solvent A: 0.01% FA in water and solvent B: 0.01% FA Acetonitrile. Samples are monitored with UV diode array, ELSD, and Single Quad ESI MS in positive and negative mode. To identify putative AZT molecules in the extracts, MS chromatograms of cultures with and without added D10-leucine were compared and scanned for peaks in the molecular weight range of 700-1200 Da having a shift of +8 Da in the presence of D10-leucine. Initial target ID experiments in the WT AZT010 strain failed to detect labeled compounds (for example, see FIG. 2B, left panel, compare top (“SG-AZT010-WT”) and bottom (“SG-AZT010-PIJ10257-SARP”); see also FIG. 3, bottom panel). Follow-up strain engineering (next section) resulted in the detectable expression of a labeled peak with a mass of 855 [M−H] at RT 12.4 minutes (see FIG. 2B, left panel, bottom SG-AZT010-PIJ10257-SARP).

### Example 3. Overexpression of SARP Regulator (in trans) in WT Streptomyces griseochromogenes (AZT010) Strain

SARP (Streptomyces Antibiotic Regulatory Protein) regulators are a class of BGC specific regulators that have been shown to act as positive regulators of compound expression in many species of Streptomyces. AZT010 BGC harbors a SARP, aztT10, located in the middle of the NRPS and PKS core genes. To test if overexpression of this regulator affects AZT010 compound production and enable target identification, aztT10 was cloned into the integrative plasmid, PIJ10257, under the control of constitutive ermE* promoter. The resulting strain, SG-AZT010-PIJ10257-SARP, harboring a copy of the SARP gene was grown for compound production in the presence of D10-leucine as described above. Analysis of the peaks in the extracts of WT SG-AZT010, AZT010 transformed with empty PIJ10257 vector, and SG-AZT010-PIJ10257-SARP showed an overexpressed, D10-Leucine labeled peak at RT 12.4 min with m/z 855 [M-H] (See FIG. 2B, left panel, bottom)

Cloning of PIJ10257-SARP construct. SARP gene, aztT10, was amplified from genomic DNA of AZT010 producer using primers (azt010-PIJF/R) containing overlaps for Gibson assembly with PIJ10257 plasmid. PIJ10257 plasmid was digested with HindIII for 2 hrs at 37° C. and purified by precipitation. The purified linear plasmid was assembled with the aztT10 PCR fragment using Infusion cloning kit (Takara), transformed into Stellar competent cells (Invitrogen), and plated on LB agar with apramycin (50 ug/mL). Plates were incubated at 30° C. overnight. Colonies were picked into 5 mL LB broth with apramycin (50 ug/mL) and grown overnight at 37° C., 200 rpm. Resulting constructs (PIJ10257 -SARP) were analyzed by restriction digest and verified by sanger sequencing.

Conjugation of PIJ10257-SARP into AZT010 producer. Verified PIJ10257-SARP plasmid was transformed into ET12576/puz8002 E. coli strain by electroporation. Colonies were selected on apramycin (50 ug/mL) and conjugated into AZT010 WT strain. The receiving AZT010 WT spores were prepared by streaking on MSF agar (mannitol 20 g/L, soy flour 20 g/L, CaCl2 10 mM) and incubating the plates for 7-10 days at 28° C. Recovered spores were aliquoted into 30 uL volumes (approximate spore titer of 10⁹ cells/mL) and stored at −80 ° in 10% glycerol. Exconjugants selected on apramycin (50 ug/mL) /nystatin (30 ug/mL)/nalidixic acid (50 ug/mL) plates were observed after 4-6 days of growth at 30° C. Colonies were picked into 5 mL TSB with apramycin (50 ug/mL) and grown for 3-4 days at 28° C., 220 rpm. Screening primers (pij-azt010-sc-F/R) were used to select for edited strains which were further verified by sanger sequencing.

Small scale production and identification of target peak. Small scale growth and production was carried out in a matrix of conditions using 5 different production media and 4 different time points. The experiment was performed with triplicates using 50 mL media in 250 mL baffled flasks. Glycerol stocks of SG-AZT010-PIJ10257-SARP strain, SG-AZT010-PIJ10257 plasmid, and SG-AZT010 WT strain were each scraped and inoculated into 10 mL TSB apramycin (50 ug/mL), and grown at 28° C., 220 rpm for 3-4 days. 500 uL of this seed culture was used to inoculate each 50 mL flasks with different media. D10-leucine was added at inoculation. The cultures were grown at 28° C., 220 rpm. Subsets of cultures were harvested at 6, 8, 10, and 14 days, extracted, and analyzed in the LCMS (see extraction and LCMS analysis methods section). Chromatograms were analyzed for peaks that are overexpressed/present in SG-AZT010-PIJ10257-SARP compared to the wild type strain. The mass spectra were then examined for the presence of a mass shift indicating the incorporation of β-OH D10-leucine. Two peaks with the same mass—m/z 855 [M-H], eluting at RT of 12.4 and 12.6 mins were identified as products of the AZT010 BGC (FIG. 2B, bottom left panel).

### Example 4. Heterologous Production of AZT010 BGC in S. albus J1074

Transfer of AZT010 BGC into S. albus J1074. To confirm that the m/z 855 [M-H] target peak(s) is a product of AZT010 BGC and produce the compounds in a tractable host, the 120 kbp region spanning the core NRPS-PKS gene cluster and surrounding genes was cloned from the chromosome of AZT010 WT. The cloned region contains genes related to precursor biosynthesis, post-pks tailoring, regulation, and transport in addition to the core NRPS-PKS. Cloning was performed by a combination of in vitro CRISPR Cas9 digestion at the specified edges of the gene cluster, and subsequent Gibson assembly with pDualP BAC (Varigen Biosciences). The cloned BGC was sequenced using a combination of long-read and short-read sequencing and conjugated into S. albus J1074 producing the heterologous strain SA-pDualP-AZT010 (FIG. 3). Analysis of extracts from the cultures of SA-pDualP-AZT010 showed production the target peak(s), confirming that m/z 855 [M−H] peaks are products of the AZT010 BGC.

Conjugal transfer of AZT010 into S. albus. For conjugation of the BGC into S. albus, purified pDualP-AZT010 plasmid DNA was transformed into E. coli S17 cells by electroporation. Colonies were grown at 30° C. overnight under apramycin (50 ug/mL) and trimetropim (10 ug/mL) selection. Colonies were picked into 5 mL of the LB broth and grown overnight at 30° C. under the same antibiotic selection. Overnight cultures were screened for the presence of the BGC using 3 primer sets spanning the gene cluster, as well as a primer set designed for the junction of the backbone plasmid and the BGC. For conjugation, 200 uL of an overnight grown E. coli S17 cells containing the AZT010 BGC was inoculated into 50 mL of LB broth with antibiotics and grown at 37º to an OD600nm of (0.6-0.9). Cells were washed 3× with 20 mL of LB and resuspended in 500 uL SOC. To prepare the receiving S. albus strain, 30 uL of spores (stocked at 10×9 CFUs) was diluted into 1 mL of SOC, heat shocked at 50° C. for 10 min, and cooled at room temperature. 100 uL of the washed E. coli cells were mixed with 200 uL of heat shocked spores. 200 uL of the mating mixture was spotted on ISP4-AMC plates containing nystatin (30 ug/mL) and incubated at 30 o for 16 hours. Grown mating spots were scraped into LB and plated on ISP4-AMC plates with nalidixic acid (50 ug/mL) and nystatin (30 ug/mL) and apramycin (50 ug/mL) and incubated at 30° C. for another 2-4 days. S. albus ex-conjugants were picked into 10 mL of TSB broth containing apramycin (50 ug/mL) and grown for 2 to 3 days in a shaking incubator at 30° C. 220 rpm. Cultures were screened by PCR using the BGC screening primers described above to confirm integration. Confirmed positive strains (SA-pDualP-AZT010) were glycerol stocked and stored for later production studies.

Small scale production, extraction, and analysis. Small scale production from the SA-pDualP-AZT010 and SA-pDualP was performed using the procedure and conditions described above. The production media used was R5A (Sucrose: 100.0 g/L, K2SO4: 0.25 g/L, MgCl2: 10.12 g/L, Glucose: 10.0 g/L, Casamino Acids: 0.1 g/L, Yeast Extract: 5.0 g/L, MOPS: 21.0 g/L, pH 6.85, trace elements: 2 mL/L) and cultures were grown for 7 days before extraction. Extracts were analyzed by comparing the LCMS chromatograms of SA-pDualP (empty vector control) and SA-pDualP-AZT010. Production of the target peaks m/z 855 [M−H] was confirmed from the SA-pDualP-AZT010 extract, confirming that these peaks are products of the AZT010 BGC.

### Example 5. Titer Improvement in S. albus J1074

Promoter engineering. To increase the production titer of target peaks and enable isolation and characterization AZT010 in S. albus, a series of known, strong promoters were designed for scarless insertion in the AZT010 BGC by in vivo CRISPR-Cas9 editing (Cobb 2014, Zhang 2017) including ermE*, kasO*, gapdh, and rpslp promoters. Three sites were chosen: 1) upstream of the mbtH gene driving the NRPS operon, 2) in between the NRPS and PKS regions (bidirectional), and 3) downstream of the PKS in front of aztQ precursor biosynthesis gene (FIG. 3).

Cloning of editing template. A gene-synthesized editing plasmid, pCrispomyces2, was used for scarless editing of BGCs in vivo. Editing plasmids were constructed in two steps 1) insertion of 20 nt protospacer sequences for the target region into the BbSI site by Golden gate assembly, and 2) cloning of the editing fragment containing the desired promoter in between two homologous arms into the Xbal site. Three to five sgRNAs cut sites were designed within a 500-1000 bp of the desired region. Protospacer oligos were annealed and cloned into the plasmid by Golden gate assembly as described (Cobb 2014). The resulting plasmid containing sgRNA recognition sequences were then used as templates for the next step. 1000 bp homologous regions upstream and downstream of the target region for promoter insertion were amplified with primers containing overlaps for 4-way Gibson assembly with the plasmid resulting from step 1, and a PCR-amplified promoter fragment. The resulting editing plasmids containing an sgRNA protospacer sequence under the control of gapdh promoter, and an editing fragment containing 5′-left homologous region-promoter-right homologous region were verified by restriction digests and sanger sequencing.

Promoter knock-in in SA-pDualP-AZT010 BGC. Sequence verified editing plasmids were transformed into ET12567/puz8002 E. coli cells and conjugated into SA-pDualP-AZT010 strains using the method described with some modifications. Conjugants were grown on the same antibiotics above with the addition of hygromycin (100 ug/mL) to select for the editing plasmid. After 3-4 days, ex-conjugants were re-streaked on plates without hygromycin and incubated at 37° C. to cure the editing plasmid. 10-20 colonies were picked from each editing experiment and grown in 5 mL TSB for 2-3 days at 28° C., 220 rpm. Each culture representing individual colonies were then screened by PCR, targeting the edited regions. When necessary, additional purification was performed by re-streaking of the colonies and subsequent PCR screening. For the insertion of kasO* upstream of the MbtH, 3/12 colonies screened positive for the desired editing. Edited SA-pDualP-AZT010 strains were glycerol stocked. Small scale production comparing the titers of different strains was performed as described. Peaks were quantified as AUC at UV282 nm and calculated using AZT010 pure compound standard curve. From the series of edited strains, SA-pDualP-AZT010-kasO* showed consistently increased titers (up to 10 mg/mL) compared to SA-pDualP-AZT010 only and other edited strains (FIG. 2B). This strain was chosen for LS production of the target molecule(s).

Construct sequences used to generate the strains in this example are shown in Table 4 below:

TABLE 4
Constructs

| | SEQ ID |
|---|---|
| Name | NO: |
| PIJ10257-SARP-aztT10 (vector sequence with SARP gene | 37 |
| under ermE*p used to integrate into WT AZT010 chromosome) | |
| PIJ10257-SARP-aztT10-hyg (vector sequence with SARP gene | 38 |
| under ermE*p used to integrate into SA-pDualP-AZT010 | |
| chromosome) | |
| PIJ10257-SARP-trns2 (vector sequence with SARP gene and | 39 |
| transporters under ermE*p used to integrate into SA-pDualP- | |
| AZT010 chromosome) | |
| pC2hyg-sg4-AZT010-kasO-editing (editing pCrispomyces2 | 46 |
| plasmid for inserting kasO* promoter into SA-pDualP-AZT010) | |

### Example 6. Large Scale Production and Isolation

Large scale production. 0.5 mL of 2-3 days old seed cultures prepared as described above was inoculated into 50 mL R5A media in 250 mL baffled flasks. A total of 5-10 L of cultures were grown in batches at 28° C., 220 rpm, and 7 days. At day 7, the cultures (mixed mycelia and broth) were extracted with equal volumes of 1:1 IPA: chloroform twice. The extracts were dried under vacuum to yield the crude material (˜3-10 grams).

Isolation and purification. Crude extract (3.66 g) from a 5L culture of SA-pDualP-AZT010-kasO* was subjected to silica flash chromatography (ISCO, Teledyne) on a Redisep column (80g) using the following conditions: flowrate 60 ml/min; step gradient: 0-10 min 100% A, 10-20 min 100-0% A, 20-30 min 100% B. Solvent A: chloroform, and Solvent B: methanol. Fractions were automatically collected by peak and volume and monitored at UV282 nm. Fractions were profiled in the LCMS and those containing the target mass were pooled, dried, and subjected to a second flash chromatography fractionation on a C18 Redisep column (50 g). The conditions for the second step are the following: flowrate: 40 mL/min; step gradient: 0-5 min 30% B, 5-35 min 30-60% B; 35-40 min 60 -100% B, 40-50 min 100% B. Solvent A: water and Solvent B: acetonitrile. 13 fractions were collected by peak and volume as monitored at UV 282 nm and subsequently profiled by LCMS. F7 to F11 contained the target peaks with m/z 855 [M−H] and were combined for final HPLC purification on a Luna C18 semipep (100 A, 250×4.6 mm) column. The following HPLC conditions were used: flowrate 4.5 mL/min; isocratic gradient at 50% B for 45 min. Solvent A: 0.1% FA in water with, and Solvent B: 0.1% FA acetonitrile. 15 and 7 mg of m/z 855 [M−H] peaks 1 and 2 were isolated respectively.

### Example 7. Compound Characterization and Planar Structure of AZT010 Compound, Formula (I)

AZT010 compound 1 peaks were isolated as white powder with UV lambda max at 210 and 282 nm. HR ESIMS analysis gave an m/z 857.4761 [M+H]⁺ and 879.4587 [M+Na]⁺ for both peaks and a molecular formula of C42H64N8O11 (calculated for 857.4761 [M+H]+).

¹H NMR spectrum of peak 1 in CDCl₃ is consistent with a peptidic molecule with ¹ H chemical shifts at 4-5 ppm for alpha protons. Distinct chemical shifts shown for AZT-like molecules are also present such as the ¹H at 5.48 ppm for the beta proton of b-OH leucine and a downfield singlet at 9 ppm for the N—OH proton. The full chemical structure (FIG. 3) was determined by a combination of 2D NMR (gHSQC, gCOSY, gTOCSY, HMBC, NOESY) assignments in CDCl₃ and HR ESIMS/MS fragmentation analysis. The presence of β-OH leucine residue was confirmed by HMBC and COSY cross peaks between the alpha, beta positions and the two doublet CH3s of the sidechain. Serine was assigned based on HMBC correlations of the amide NH and alpha proton to the OH linked beta carbon at 59 ppm. 2 piperazic acid residues were assigned from ¹H COSY/TOCSY spin systems with chemical shifts consistent with reported literature, including the unusual NH at 4-5 ppm. The presence of rare non-proteinogenic unsaturated cyclohexyl-alanine residue was confirmed by HMBC correlations between the CH₂ protons of the ring and the double bond CHs at 5.5 (dq, J=9.9) and 5.7 (dd, J 9.6) ppm with corresponding HSQCs at 127.4 and 129 ppm and vise versa. HMBC cross peaks between the N—CH₃ and carbonyl carbons of cyclohexyl-alanine and N—OH alanine supports N-methylation at this position. The sequence of the core peptide (ser-piz-(Nme)cyclohexylala-(NOH)ala-piz-(bOH)leu) was determined by key HMBC correlations and further supported by MS/MS fragmentation in both ESI positive and negative mode.

The polyketide tail of AZT010 compound 1 is unique among the AZT molecules. While HMBC confirms the amide linkage to the β-OH leucine residue, it lacks the canonical tetrahydropyran ring typified by the anomeric carbon at 95-99 ppm which is missing in the HSQC spectra. Instead, HMBC-COSY-TOSCY correlations reveal and conjugated system with di-ketide-diene tail. Based on the planar structure, the two-peak (multiple peak) behavior of the target compound maybe explained by tautomerism and conjugation at C34.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

What is claimed is:

1. A compound of Formula (I):

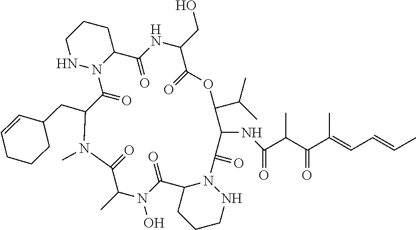 [SMILES: C/C=C/C=C(\C)C(=O)C(C)C(=O)NC1C(=O)N2NCCCC2C(=O)N(O)C(C)C(=O)N(C)C(CC2C=CCCC2)C(=O)N2NCCCC2C(=O)NC(CO)C(=O)OC1C(C)C]

2. The compound of claim 1, wherein the compound is produced by a host cell comprising a heterologous biosynthetic gene cluster comprising at least six nonribosomal peptide synthetase (NRPS) modules and at least four polyketide synthase (PKS) modules, a set of modifying enzymes, precursor biosynthesis enzymes, transporters, and one or more transcriptional regulators.

3. The compound of claim 2, wherein the biosynthetic gene cluster is isolated or derived from Streptomyces griseochromogenes strain ATCC 14511.

4. The compound of claim 2 of claim 3, wherein the biosynthetic gene cluster comprises a sequence of SEQ ID NO: 1.

5. The compound of any one of claims 2-4, wherein the biosynthetic gene cluster comprises one or more modifications of SEQ ID NO: 1.

6. The compound of any one of claims 2-5, wherein the modification comprises a substitution, deletion, inversion, or insertion of one or more nucleotides relative to SEQ ID NO: 1.

7. The compound of claim 5 or claim 6, wherein the modification comprises insertion of at least one promoter sequence.

8. The compound of claim 7, wherein the promoter is selected from the group consisting of ermE, kasO, gapdh, and rpslp, or functional variants or derivatives thereof.

9. The compound of any one of claims 5-8, wherein the modification increases synthesis of the compound of Formula (I) compared to an otherwise equivalent host cell comprising an unmodified biosynthetic gene cluster.

10. The compound of any one of claims 2-9, wherein the host cell is a Streptomyces albus cell.

11. The compound of any one of claims 2-10, wherein the host cell further comprises a sequence encoding a Streptomyces Antibiotic Regulatory Protein (SARP) operably linked to a constitutive promoter.

12. A polynucleotide comprising a biosynthetic gene cluster, wherein the biosynthetic gene cluster comprises one or more genes that contribute to the production of at least a portion of the compound of claim 1 when the biosynthetic gene cluster is expressed by a host cell

13. The polynucleotide of claim 12, wherein the one or more genes comprise six nonribosomal peptide synthetase (NRPS) modules.

14. The polynucleotide of claim 13, wherein the six NRPS modules are encoded by sequences comprising a first NRPS open reading frame of SEQ ID NO: 14, a second NRPS open reading frame of SEQ ID NO: 15, a third NRPS open reading frame of SEQ ID NO: 16 and a fourth NRPS open reading frame of SEQ ID NO: 17, or sequences having at least 90%, at least 95%, at least 97% or at least 99% identity thereto.

15. The polynucleotide of any one of claims 12-14, wherein the one or more genes comprise four polyketide synthase (PKS) modules.

16. The polynucleotide of claim 15, wherein the four PKS modules are encoded by sequences comprising a first PKS open reading frame of SEQ ID NO: 26 and a second PKS open reading frame of SEQ ID NO: 27, or sequences having at least 90%, at least 95%, at least 97% or at least 99% identity thereto.

17. The polynucleotide of any one of claims 12-16, wherein the biosynthetic gene complex comprises a Streptomyces Antibiotic Regulatory Protein (SARP)-encoding gene.

18. The polynucleotide of claim 17, wherein the SARP-encoding gene comprises a sequence of SEQ ID NO: 28, or a sequence having at least 90%, at least 95%, at least 97% or at least 99% identity thereto.

19. The polynucleotide of any one of claims 12-18, wherein the biosynthetic gene cluster comprises a sequence of SEQ ID NO: 1, or a sequence having at least 90%, at least 95%, at least 97% or at least 99% identity thereto.

20. The polynucleotide of any one of claims 12-19, wherein the host cell is engineered to express the one or more genes in the biosynthetic cluster, which results in the production of the compound of Formula (I).

21. The polynucleotide of any one of claims 12-19, wherein overexpression of one or more genes in the biosynthetic cluster by the host cell increases the production of the compound of Formula (I) compared to an otherwise equivalent host cell comprising a biosynthetic gene cluster that does not overexpress one or more genes in the biosynthetic cluster.

22. The polynucleotide of claim 20 or claim 21, wherein the SARP is overexpressed.

23. The polynucleotide claim 22, wherein overexpression of the SARP occurs in cis or in trans.

24. The polynucleotide of claim 23, trans overexpression of the SARP comprises expressing a sequence encoding the SARP open reading frame under the control of a constitutive ermE promoter, or a functional variant or derivative thereof.

25. The polynucleotide of claim 24, wherein the ermE promoter comprises a sequence of SEQ ID NO:33.

26. The polynucleotide of any one of claims 12-15, wherein the biosynthetic gene cluster comprises one or more sequence modifications relative to a biosynthetic gene cluster of SEQ ID NO:1, or a sequence having at least 95%, at least 97% or at least 99% identity thereto.

27. The polynucleotide of claim 26, wherein the one or more modifications of the biosynthetic gene cluster comprises a substitution, deletion, inversion, or insertion of one or more nucleotides relative to SEQ ID NO: 1.

28. The polynucleotide of claim 26 or claim 27, wherein the one or more modifications comprise modifications of a promoter of a gene in the biosynthetic gene cluster.

29. The polynucleotide of claim 26 or claim 27, wherein the one or more modifications comprise insertion of at least one heterologous promoter in the biosynthetic gene cluster.

30. The polynucleotide of claim 29, wherein the at least one heterologous promoter is a strong promoter.

31. The polynucleotide of claim 29 or claim 30, wherein the at least one heterologous promoter is selected from the group consisting of ermE, kasO, gapdh, and rpslp, or functional variants or derivatives thereof.

32. The polynucleotide of claim 31, wherein the sequence of the ermE promoter comprises SEQ ID NO: 33, the sequence of the kasO promoter comprises SEQ ID NO:34, the sequence of the gapdh promoter comprises SEQ ID NO:35, and the sequence of the rpslp promoter comprises SEQ ID NO:36, or sequences having at least 90%, at least 95%, at least 97% or at least 99% identity thereto.

33. The polynucleotide of any one of claims 29-32, wherein inserting the at least one heterologous promoter into the biosynthetic gene cluster comprises a nucleic acid guided endonuclease.

34. The polynucleotide of claim 33, wherein the nucleic acid guided endonuclease is in a complex with at least one guide nucleic acid (gNA).

35. The polynucleotide of claim 33 or claim 34, wherein the nucleic acid guided endonuclease is a CRISPR/Cas endonuclease.

36. The polynucleotide of claim 35, wherein the CRISPR/Cas endonuclease is Cas9.

37. The polynucleotide of any one of claims 29-36, wherein inserting the at least one heterologous promoter into the biosynthetic gene cluster further comprises a donor template comprising a sequence of the heterologous promoter.

38. The polynucleotide of any one of claims 29-37, wherein the biosynthetic gene cluster comprises an mbtH gene upstream of the four NRPS open reading frames, and wherein the at least one heterologous promoter is inserted upstream of the mbtH gene.

39. The polynucleotide of claim 38, wherein the at least one heterologous promoter is a kasO promoter.

40. The polynucleotide of claim 38 or claim 39, wherein the targeting sequence of the at least one gNA comprises SEQ ID NOS: 40-44, or a sequence having at least 80%, at least 85%, at least 90%, or at least 95% thereto.

41. The polynucleotide of claim 39 or claim 40, wherein the biosynthetic gene cluster comprises a sequence of SEQ ID NO: SEQ ID NO: 49.

42. The polynucleotide of any one of claims 29-37, wherein the at least one heterologous promoter is inserted between the sequence of the SARP-encoding gene and the first PKS open reading frame.

43. The polynucleotide of any one of claims 29-42, wherein the biosynthetic gene cluster comprises an ornithine monooxygenase gene downstream of the second PKS open reading frame, and wherein the at last one heterologous promoter is inserted downstream of the second PKS open reading frame and upstream of the ornithine monooxygenase gene.

44. The polynucleotide of any one of claims 12-27, wherein the at least one modification of the biosynthetic gene cluster comprises a modification that results in overexpression of the SARP-encoding gene in comparison to the expression of the SARP-encoding gene by the biosynthetic gene cluster of SEQ ID NO: 1.

45. The polynucleotide of any one of claims 12-27, wherein the at least one modification of the biosynthetic gene cluster comprises replacement of at least one promoter in comparison to the biosynthetic gene cluster of SEQ ID NO: 1.

46. The polynucleotide of claim 45, wherein replacement of the at least one promoter comprises replacement a SARP-encoding gene promoter.

47. The polynucleotide of claim 46, wherein the SARP-encoding gene promoter is replaced with a promoter selected from the group consisting of ermE, kasO, gapdh, and rpslp.

48. The polynucleotide of any one of claims 12-28, wherein the biosynthetic gene cluster comprises a sequence of SEQ ID NO: 50.

49. The polynucleotide of any one of claims 12-48, wherein the biosynthetic gene cluster is isolated or derived from Streptomyces griseochromogenes strain ATCC 14511.

50. The polynucleotide of any one of claims 12-49, wherein the biosynthetic gene cluster produces the compound of Formula (I) in the host cell.

51. A vector comprising the polynucleotide of any one of claims 12-50.

52. The vector of claim 51, wherein the vector is a bacterial artificial chromosomal vector.

53. The vector of claim 51 or claim 52, wherein the vector further comprises at least one promoter.

54. The vector of any one of claims 51-53, wherein the vector is suitable for expression in a Streptomyces species cell.

55. A host cell comprising the polynucleotide of any one of claims 12-50 or the vector of any one of claims 51-54.

56. A host cell, comprising the polynucleotide of any one of claims 12-50 and a polynucleotide comprising a sequence encoding a SARP operably linked to a constitutive promoter.

57. The host cell of claim 56, wherein the constitutive promoter is an ermE promoter.

58. The host cell of claim 56 or claim 57, wherein the SARP is encoded by a sequence of SEQ ID NO: 28.

59. The host cell of any one of claims 55-58, wherein the host cell is an Actinobacterial cell.

60. The host cell of any one of claims 55-59, wherein the host cell is a Streptomyces cell.

61. The host cell of claim 60, wherein the Streptomyces cell is a Streptomyces griseochromogenes, Streptomyces lividans or Streptomyces albus cell.

62. A method of making a polynucleotide comprising a modified biosynthetic gene cluster comprising:
a. providing a first E. coli host cell comprising a first vector comprising a sequence of an unmodified biosynthetic gene cluster comprising a target sequence;
b. introducing the first vector into a Streptomyces host cell by conjugation;
c. providing a second E. coli host cell comprising a second vector comprising:
i. a sequence of at least one gNA specific to the target sequence operably linked to a promoter,
ii. a sequence encoding a Cas9 endonuclease; and
iii. a sequence encoding a donor template; and
d. introducing the second vector into a Streptomyces host cell by conjugation; whereby introducing the second vector into the Streptomyces host cell produces a double strand break in the target sequence and introduction of a donor template sequence, thereby generating a Streptomyces host cell comprising a modified biosynthetic gene cluster.

63. The method of claim 62, wherein the unmodified biosynthetic gene cluster comprises a sequence of SEQ ID NO: 1.

64. The method of claim 62 or 63, wherein the donor template comprises, from 5′ to 3′, a sequence homologous to a sequence 5′ of the target sequence, a sequence of a promoter, and sequence homologous to a sequence 3′ of the target sequence.

65. The method of claim 64, wherein the promoter is selected from the group consisting of ermE, kasO, gapdh, and rpslp, or functional variants or derivatives thereof.

66. The method of any one of claims 62-65, wherein the at least one gNA comprises a target sequence selected from the group consisting of SEQ ID NOS: 40-44.

67. A method of making the compound of Formula (I), comprising
a. introducing into a host cell the polynucleotide of any one of claims 12-50 or the vector of any one of claims 51-54;
b. culturing the host cell under conditions sufficient for the synthesis of the compound of Formula (I) by the biosynthetic gene cluster; and
c. isolating and purifying the compound of Formula (I).

68. The method of claim 67, wherein the host cell is an Actinobacterial cell or a Streptomyces cell.

69. The method of claim 68, wherein the Streptomyces cell is a Streptomyces griseochromogenes, Streptomyces albus or Streptomyces lividans cell.

70. The method of any one of claims 67-69, wherein the host cell comprises a sequence encoding a SARP operably linked to a constitutive promoter.

71. The method of any one of claims 67-70, wherein the polynucleotide or vector is introduced into the host cell by conjugation with an E. coli comprising the polynucleotide or vector.

72. A pharmaceutical composition, comprising the compound of claim 1, and a pharmaceutically acceptable excipient.

73. A method of treating a disease or disorder in a subject, comprising administering the compound of claim 1 or pharmaceutical composition of claim 72.

74. A compound of claim 1 or the pharmaceutical composition of claim 72, for use in treating a disease or disorder in a subject.

75. A compound of claim 1 for use in the manufacture of a medicament for treating a disease or disorder in a subject.

76. Use of a compound of claim 1 or the pharmaceutical composition of claim 72, for the treatment of a disease or disorder.

77. The method, use, or compound of any one of claims 73-76, wherein the disease or disorder is cancer.

78. The method, use, or compound of any one of claims 73-76, wherein the disease or disorder is fibrosis.

79. The method, use, or compound of any one of claims 73-78, wherein the subject is human.
